SEQUENCE LISTING (34 sequences)

SEQ ID NO: 1 - DNA, 194 bp, Ixodes ricinus
ataccttccacttgtagcccttcctcatccgatatggtgacggatgccattgcatcctcgtcgtggaagaggtcctcttctaaataagacccatccatatatgtgtgtttgcgaatgccgtcgacgtagctcctgactagaaactcgtcggctaggacagaacttttcttcaggtttagcgtaatgtcctcgtt

SEQ ID NO: 2 - DNA, 607 bp, Ixodes ricinus
  misc_feature (5): n=A,C,T or G
taccngggaatccaaaaccaatttttattggaacttccacgtcttcttcaaggcggtggcacctctgcatttatgaagttcgtcttggcattttattttttgcttctttcattgcrgaactcgcaaatgcacttcccgtgcttgtcgcatttcgccccaaaagcgcatggcattccttccggcagattaactttttcaaattcacggttctgaaccaataatagatcgtggcaatgtttgtgctgtttgcgatttgcaaaccagctgtagccaccattggactcaaaggtgcgcacaacatggcgccgaactgtgaaaaacaaattaaggctnctttgtaataacgctagtcttggtacgccgttagaggtcgatgtcgcgcctcgcgattgcaaagtcacttgcacttatcaagctcctggagaaaaatgggtgcaacggggggatcagcgtttgtacttgcaaacatttgtggagacggtaaaccwgtatttcgcggaactcagatgctccagcgtgaagctcgtcttaataaaagttgtaaattcgagtatngatgaagaactgaaattcgaggcatttagaaacaccacgagaagcagcggaa

SEQ ID NO: 3 - DNA, 259 bp, Ixodes ricinus
gatcctacgcctgaaaatgagtgtccatcgtcttcacatagtgccacattgtaattggtacaagctccattttcgtcagcgctgtttgttatgctgccgcctacttttccttcggcactccataagttaaaccctgtcattataagtgtgattgccgtatctcggctgaatgggttccatttttctcttaaataatcacgtgtccatattccatgtattgtgttcatgagtatgtgattctcatcgtatatcttcgcct

SEQ ID NO: 4 - DNA, 170 bp, Ixodes ricinus
ccactcgaaaatggaggctttgaaacatttcagtacccctgtgaactctggctttgcaatgtaacagcaaaaacacttacagttgaagggtgcagtgtcagacgctatggaagttgcatccacgagcacraccctgattactactggccacgttgctrtccgggtcgtcc

SEQ ID NO: 5 - DNA, 168 bp, Ixodes ricinus
gtatgttaccatgtccaacccggttattaaatacaccaagtcgtaggatttgtaggcagctgcattgcccttgacgtactctctcaacgttgccaaggactcaggcccataaatgtagtggggttgaccttgaactcttcgtaaaaagcgttctttctccgtcgtgag

SEQ ID NO: 6 - DNA, 247 bp, Ixodes ricinus
ccgaamataaaacttagtctcaccaatatacgtttgcctaacgcgaaggaacaggcacaaatatactacgagcacgacattctcaagaacacggttcacggagtgtggacgagaattcactcaaaatatccgttccctgaagatgagggaattacactgataatgacagggtttgatttatggagtgccgatttaactgtaggcggcaccataacaaacagcgctgagaaaagcggagcttgtacga

SEQ ID NO: 7 - DNA, 261 bp, Ixodes ricinus
  CDS (1)..(258)
atgccttttattttcgtggtgagcttagtcattgtggcctgcatcgtggtagacacagccaaccacaaaggtagagggcggcctgcgaagtgtaaacttcctccggacgacggaccatgcagagcacgaattccgagttactactttgatagaaaaaccaaaacgtgcaaggagtttatgtatggcggatgcgaaggaaacgaaaacaattttgaaaacataactacgtgccaagaggaatgcagagcaaaaaaagtctag

SEQ ID NO: 8 - protein, 86 aa, Ixodes ricinus
MPFIFVVSLVIVACIVVDTANHKGRGRPAKCKLPPDDGPCRARIPSYYFDRKTKTCKEFMYGGCEGNENNFENITTCQEECRAKKV

SEQ ID NO: 9 - DNA, 292 bp, Ixodes ricinus
catcgmagccatagtatattttgcacttgtcttccgtttcgtcgtagtaggaccgattccacattgtagtacaccagtcacttatatcctgcgggcggtgcttgcatttgtcctgaacaaatcttccacagcgcttgtcgcacgcctcctgggaatagaacgcgttctctcctccgcatctccatttggaatcatagaaacatctttcagtttgaatattgtagcgataataatcggtatcagtttctttgcatggtcctgggaggggtttggcgcaggggccgtattcagg

SEQ ID NO: 10 - DNA, 270 bp, Ixodes ricinus
ggtaatagttgtcaaattccattaatgtatcctgaaatgtgaccatatctttgtttcccctgtaaaatctcataaaaggctgtgtgttttccttaagaagtgtaacagccacgatggtcaatctcacggatggatgtgtgacacttttatatctcaggtttgccgacattgccattacagataaatagttgataatttctttcttgttatagttgtaagcagcgcatgttgttgcatcaagcaccacatgcacttcaggcaatatggttt

SEQ ID NO: 11 - DNA, 316 bp, Ixodes ricinus
agaaagcagtcatattggccatccacaggtcacaatggttctctccttgacctggcatcgggattcgaagtatggtgcagttcacgtagttggaatacaacacgaaatgtgttcgttggtacgccaataggggttctcgcaaagaacatatcatttggaggaaggcgtagtccgtcgagatatcccaaaactagggtttcattgcgtgcgaaccaactgcccccacttctgtatgtgtactgtaaggagtrgttgaacggygtcctctttcccataaccttgaagttttcacactgcagaggattacctctcaaaa

SEQ ID NO: 12 - DNA, 241 bp, Ixodes ricinus
aaggtagcaagggtggtaggctttcctcacaaagagtctggcttccgtgataaccatatccattcctcaccgtatacccgtcatccaacgtcaattgtgttacaaggcagataatgtcaaaatggctctggtccctataatagtcggataatgtagaaatcgctccatgtggccaaatagatgttcctctttcatactgttttaactttaattgtaggtccgcctcgttctcgaggtatgt

SEQ ID NO: 13 - DNA, 636 bp, Ixodes ricinus
  misc_feature (7): n=A,C,T or G
ttccccnaattggccttgcganncttgcaagtcgacnctagaggctccgaagatggacagattgcgcatgaaatatttgaaatcgagcagaatggtgattttaggagcgattatattgtgccacccagtttgaaagtgcaagaacgcacagtggtttaccgtaacaagtacaccagagttcctgtaaattttaccgtcgaagttgccatgctgattgataagtatttatacwaggagttcaagaacgagagccacatcgtaccgtacctggctatgatactgactttgataaatctgaggtatgccgacacacatgacccgtacatccagtttcttctcacacaagtgttcgtggggaawwctggcgatcatatgggccacatgcccttccgacgagcgttcttgttcaggcgccggcattatgcgcagtttaggcccaatmacaccttccacttgtaattctccgttgttggatagtgtaagtgaggccattgcatcagcatcgtggaagargccttcctccaagtaggaaccgcccatttaggtttgctttcccaatccgccaatttaanttttaaaaaaaattccccccccaaaaattaattttttttaaaggtggattgtgatttctccgtt

SEQ ID NO: 14 - DNA, 432 bp, Ixodes ricinus
gatcccaaaagtgcccctggarcgacggttacatcatgagctacgtcataaacttcaaaaaccacttcaaattttctccctgctgtgtagaatcaattcgattcgtcgcacgagagcgggactgcctctacaaagtcaatgccaaggatgctgtaaaaagcctaatatctctgcccggatttaggatatcgccaacgagtttctgtcaatttatgcatccgctttaccgcggtgtccatagcgataagaaagcaggtctgtccgattgcgtacagacgtgtagaacggccaaaaatcgacgaggaggctaccattcatggattcacgcggcacttgacggggttccttgcgacaagagaaaccccaagaaggcctgcataaacgggaaatgcaccctccttaagagcatgccccacagaacgtaccgggaat

SEQ ID NO: 15 - DNA, 466 bp, Ixodes ricinus
agggcgttctttgcttyacagggaacrgcatatgggccacgtgaccttccaatgaccgctccaaatctggcataggttgaaytcgcaagtcgtggcgcagcaggcctyccacattcactccatcctcgtcttttaggatgactgccgccatttgttttgtatcgtggtacaggtgtttgttatggtccgagccgtcgacataagtattgaccaacgatcggccgaatgattacggctcaccaaacacatcaaatacccccgtcaagtcaagagctggaagcacaaagcatagtatgtacaagatacccttggaaatctttcccgaagttcaccttgtggtggacagcacatttgccaaagcttttaaatttgacgtgtacaaagtaacgcgttacttcgcagtgcttacaaatgcggctaatcttaggtatgccagcttcgtatttccaaaagtacagctcaggat

SEQ ID NO: 16 - DNA, 377 bp, Ixodes ricinus
ctcgtccacacattctcctaaaatgcaagccttttttttcccacaaggtgtaccgtcgactacactgagtctccaataaatatgttttccggtgcaatttaccttgcagtctttgacgccgtatgtagggtcagcgtgcatgccttcgtcgtacatatacaccctctgacagtagttgctcagtgttgtcatcctaccaggaagcttagacgaacgttttattgtttttgtcgtgtatcgttctctaaggcatttgaattccggacggttgtagaggttcctgacttctcgctggcagcaataagagaactgatactggcgctcgtcttgcatcttgtaactcatgaggtatccgtcatcccatgggcagtccgcag

SEQ ID NO: 17 - DNA, 1670 bp, Ixodes ricinus
  CDS (54)..(1517)
aaggaagaagttaggcgtaggctttgggaaaccggtcatcctcgaaaccagagatgtcgggactcagcctgaaattgtggattgtagcgttcttttctttctgcttggccgagaaagagcatgggatcgtgtaccccaggatgcttgaaagcagagcagcaactggagagagaatgcttaaaatcaacgatgacctgacgttgacgctgcagaagagtaaggtcttcgctgacgactttctcttcagcacgaccgacggaattgaacctattgattactacatcaaagccgaagacgctgaacgtgacatctaccacgacgcaactcacatggcatcagtaagggtaacggacgatgatggcgtggaagtggaaggaattcttggagagaggcttcgtgttaaacctttgccggcaatggcccgcagcagcgatggcctcagaccgcatatgttgtacgaagtcgacgcacacgaaaacggccggccacatgattatggttcaccgaacacaacaaatacccccgtagagagaagagctggaggcacagaaccccagatgtacaagataccagcggaaatctatcccgaagtttaccttgtggcggatagtgcctttgccaaagaatttaactttgatgtgaacgccgttacgcgttacttcgcagtgcttacaaatgcggctaatcttaggtatgaaagcttcaaatctccaaaggtacagctcaggatcgttggcataacgatgaacaaaaacccagcagacgagccatacattcacaatatacggggatatgagcagtaccggaatattttgtttaaggaaacactggaggatttcaacactcagatgaagtcaaaacatttttatcgtactgccgatatcgtgtttctcgtgacagcaaaaaatatgtccgaatgggttggtagcacactacaatcatggactggcgggtacgcttacgtaggaacagcgtgttccgaatggaaagtaggaatgtgtgaagaccgaccgacaagctattacggagcttacgttttcgcccatgagctggcgcataatttgggttgtcaacacgatggagatggtgccaatagctgggtgaaagggcacatcggatctgcggactgcccatgggatgacggataccttatgagctacaagatggaagacgagcgccagtataagttttctccctactgccagagagaagtcaggaacctctacaggcgtccggaattcaaatgcctcactgaacgaaaagcgaaaaaaacaatccgctcgtctaagctacctggtgtgatgacatcatcgagcaactattgccggagggtgtacatgtacgaaaaaggcatgcacgccgacgaggcatatggcgtcaaggactgcagggtaaaatgcaccaccacatcaagaatgtattggctactcggtgtagtcgacggtacaccttgcggaaatggaaaggcttgcattcttgggaaatgcaggaacaaaatcaaaataagcaagaaggactgagaggttgataatatcaaattaatcatgatatttcaaccacatgacttcgtgctcaactggtagccccaaataaattttaaaaaaaatcccaatatgcgtggtagaaaaagcagcaaacaataaaacttctaaaaatgtcttgcaaaaatg

SEQ ID NO: 18 - protein, 488 aa, Ixodes ricinus
MSGLSLKLWIVAFFSFCLAEKEHGIVYPRMLESRAATGERMLKINDDLTLTLQKSKVFADDFLFSTTDGIEPIDYYIKAEDAERDIYHDATHMASVRVTDDDGVEVEGILGERLRVKPLPAMARSSDGLRPHMLYEVDAHENGRPHDYGSPNTTNTPVERRAGGTEPQMYKIPAEIYPEVYLVADSAFAKEFNFDVNAVTRYFAVLTNAANLRYESFKSPKVQLRIVGITMNKNPADEPYIHNIRGYEQYRNILFKETLEDFNTQMKSKHFYRTADIVFLVTAKNMSEWVGSTLQSWTGGYAYVGTACSEWKVGMCEDRPTSYYGAYVFAHELAHNLGCQHDGDGANSWVKGHIGSADCPWDDGYLMSYKMEDERQYKFSPYCQREVRNLYRRPEFKCLTERKAKKTIRSSKLPGVMTSSSNYCRRVYMYEKGMHADEAYGVKDCRVKCTTTSRMYWLLGVVDGTPCGNGKACILGKCRNKIKISKKD

SEQ ID NO: 19 - DNA, 158 bp, Ixodes ricinus
caccagtgatgcttattgttgcactgcacttgttgataatatccggtcgtcgaattgcacttcggaacttccactccaacttggcgagccgtggattttgacttctcgtgatgctccaccagacagttgcaggacttcagctgcctagatggagcctt

SEQ ID NO: 20 - DNA, 146 bp, Ixodes ricinus
  misc_feature (41): n=A,C,T or G
ctgttgttgaactgaaataaataacaaaaaaatcataaagntggaggaaagatgatcgantccccgccccttgacaatcgtccgataaaaaccaactatattcngtcctttttacaaacaattccaantgtctgaccgaaccgcga

SEQ ID NO: 21 - DNA, 140 bp, Ixodes ricinus
  misc_feature (3): n=A,C,T or G
ctnggacgangtcctatgacttgcgcttangtttcttagtcttcttcggtttcttctttttttgcttcggtttttcggtgggcgcaggtgtatagtcatcagtgtcggtgggcccatccgaatgagttgtcaaatgacat

SEQ ID NO: 22 - DNA, 143 bp, Ixodes ricinus
tgccgaaaaataacgatgatttgacgttgactctgcagaagagtaaggttttcaccgacagttttctgtttagcacgacgaaggataacgagcctatcgattactacgtgagagccgaagatgccgaacgagacatatatcac

SEQ ID NO: 23 - DNA, 140 bp, Ixodes ricinus
  unsure (41): A,C,T or G
tgttgctacagactcgacgtttcgagcttgctcgccatttmaagacaacgcactcacagaatatttaagtgcgttcgtgawagctgtgggcttacgattgcaggcgcttcantcaccagctgtgatattamagttcctag

SEQ ID NO: 24 - DNA, 144 bp, Ixodes ricinus
tcacgatagttgaaacgttgaaacttgaaatactcccacagtcgttggatgcttcagaactgctaagaacttcacactttgcaagaagtwccaaaatgaaagccgcgatgaccgatgatttagcttccatcttctatcacttga

SEQ ID NO: 25 - DNA, 95 bp, Ixodes ricinus
gaccaccccgtccgaacttgctaaakcaagcaatggagtgaggtgttctatgcgggttgattacaccaatggcgctgcgtggtgcgtggtgattt

SEQ ID NO: 26 - DNA, 1414 bp, Ixodes ricinus
  CDS (143)..(1273)
gtagggccgtgcaagcgaaggcagcgaaggctgcgagtgtacgtgcagttcggaagtgcaatatcctgttattaagctctaattagcacactgtgagtcgatcagaggcctctcttaacgccacattgaaaaaggatccaagatggaggcaagtctgagcaaccacatccttaacttctccgtcgacctatacaagcagctgaaaccctccggcaaagacacggcaggaaacgtcttctgctcaccattcagtattgcagctgctctgtccatggccctcgcaggagctagaggcaacactgccaagcaaatcgctgccatcctgcactcaaacgacgacaagatccacgaccacttctccaacttcctttgcaagcttcccagttacgccccagatgtggccctgcacatcgccaatcgcatgtactctgagcagaccttccatccgaaagcggagtacacaaccctgttgcaaaagtcctacgacagcaccatcaaggctgttgactttgcaggaaatgccgacagggtccgtctggaggtcaatgcctgggttgaggaagtcaccaggtcaaagatcagggacctgctcgcacctggaactgttgattcatcgacatcacttatattagtgaatgccatttacttcaaaggtctgtgggattctcagttcaagcctagtgctacgaagccgggagattttcacttgacaccacagacctcaaagaaagtggacatgatgcaccaggaaggggacttcaagatgggtcactgcagcgacctcaaggtcactgcgcttgagataccctacaaaggcaacaagacgtcgatggtcattctcctgcccgaagatgtagagggactctcagtcctggaggaacacttgaccgctccgaaactgtcggctctgctcggcggcatgtatgcgacgtccgatgtcaacttgcgcttgccgaagttcaaactagagcagtccataggtttgaaggatgtactgatggcgatgggagtcaaggatttcttcacgtcccttgcagatctttctggcatcagcgctgcggggaatctgtgcgcttcggatgtcatccacaaggcttttgtggaagttaatgaggagggcacagaggctgcagctgccactgccatacccattatgttgatgtgtgcgagatttccacaggtggtgaactttttcgttgaccgcccattcatgttcttgatccacagccatgatccagatgttgttctcttcatgggatccatccgtgagctctaaaaagcatattcttaacggcggccaatcagtctgtggagttatctcttagtcactaatgtgtaacaattctgcaatattcagcttgtgtatttcagtaacttgctagatctttgtgttgttgatgttaggcttcttgcg

SEQ ID NO: 27 - protein, 377 aa, Ixodes ricinus
MEASLSNHILNFSVDLYKQLKPSGKDTAGNVFCSPFSIAAALSMALAGARGNTAKQIAAILHSNDDKIHDHFSNFLCKLPSYAPDVALHIANRMYSEQTFHPKAEYTTLLQKSYDSTIKAVDFAGNADRVRLEVNAWVEEVTRSKIRDLLAPGTVDSSTSLILVNAIYFKGLWDSQFKPSATKPGDFHLTPQTSKKVDMMHQEGDFKMGHCSDLKVTALEIPYKGNKTSMVILLPEDVEGLSVLEEHLTAPKLSALLGGMYATSDVNLRLPKFKLEQSIGLKDVLMAMGVKDFFTSLADLSGISAAGNLCASDVIHKAFVEVNEEGTEAAAATAIPIMLMCARFPQVVNFFVDRPFMFLIHSHDPDVVLFMGSIREL

SEQ ID NO: 28 - DNA, 200 bp, Ixodes ricinus
accgtaaccaaaattgtttctttccagaagaatggttcaaacttttcaaacagatttcggaaactcttcttgcacttttaaaatccaatctacaatctttcctcgcacttctgaattgcattccagtttaccttccaagcaaacctcttttggcaactccagccgtactccatttcggcataccacagtgcatgcacttg

SEQ ID NO: 29 - DNA, 241 bp, Ixodes ricinus
cgtattctttgaagatttgtatacgaaacataaattcgtcatgcatacttttgatggttacacgacatgcgaagctgccgacaaagaagactgggaagataagaagcacctagttacggtagtgcgtggaccggataaacgaaagtacacgtttctacgcaacattctcaccttacaacggagagtgagagttagcaaaacaatgattgagctcgtacggaacatgtcctgtaggacattt

SEQ ID NO: 30 - DNA, 313 bp, Ixodes ricinus
  misc_feature (6): n=A,C,T or G
aagcanccggactacctgcttgaaaacgttgtacgggcaaacttggacggaaaactcccagatgctactccagttcctcccggaagctacacgtacgctgagaatgataacttcacctgctattccagaagtacaccgtttccggatggggtgaatgttgtataacggctgctgggtgcggaagactatgatggattacgcaaaaaagttctaaacgagttgtttcccatcccggaaagtctgctgtatgctgacatgatgcgacttgtggctaagaaagacagagttgatcacactagtggatgacctggga

SEQ ID NO: 31 - DNA, 2417 bp, Ixodes ricinus
  CDS (218)..(1492)
gtcgtagtcgtagtcgtagtcagttgcgcatgcgcggggctttcctgtctttcttgcctttctgcagtcgttcaccaacatgtggatacagctccggagatttgtaaacaaatactgcacttttaagcaagacttgatatttagatcgatatcctcctgttgtccgtcttgattaatcggctctttagggtttttagaataggcttttcggtacgagatgcccaaaggaaagaggggacccaaagcaggtggcgccgcgcgcggtggccggtgcgaggccagcctggctccgtcgtccagcgacgaggagtccaacgcagacacggcgagcgtgctgagctgcgcctcggagtctcgctgtggcagtgacggcaccgttggagacccagaagcggaggaggctgtgctgcatgacgactttgaagacaaactcaaggaggccatcgacggagcttcgcagaagagtgccaaaggacggctgtcgtgcctggaggcgattcgcaaggccttttccaccaaatacctgtacgacttcctcatggacagaccgagcacggtgtgcgacctggtggagcgtggggtgcgcaagggccgaggggaggaggcggccctgtgcgccactctcggggccctggcctgcgtccagctcggggtcggggccgaggcggacgccctgttcgacgccctgcgccagccgctctgcactttgctgcttgacggggcccaggggccctcccccagggccaggtgtgccactgccctcggcctctgctgcttcgtggtggactcggacaaccagctggtgctgcagccgtgcatggaggtgctctggcaggtggtgggtgccaaggcgggccccggctctccggtgctccaggcagcggccctgctcgcctggggcctcctgctcagcgtggctcccgtcgaccgcctgctggcgctcacgcgcacgcacctgccccggctgcaggagctgctggagagccccgacctggacctgcgcattgcggccggggaggtgatcgccgtcatgtacgagggggccagggactacgacgaggactttgaggagccctcggagtccctgtgtgcccagctgcgccagctggccacggacagccagaagtttcgggccaagaaggagcggcgccagcagcgctccaccttcagggacgtctaccgggccgtcagggagggggcctctcccgacgtgagcgtcaagtttggccgggaagtcctggaactggacacctggagtcgcaagctgcagtacgacgctttctgccagctgctgggctccggcatgaacctgcacctggccgtgaacgagctgctgagggacatctttgaactggggcaggtgctggcaaccgaggaccacattatctccaagatcaccaagttcgaaaggcacatggtgaacatggccagctgccgggcccgcaccaagacacgcaaccggctgagggacaagcgcgccgacgtggtcgcctgaacctgcggagggatgcttagctatgcactcgccggcctaccctggcgggactcgatgccactcacgagtcggcgctcgcaaattcgccgcccatcgttacgcaatgggagacaaagctgcttttggcattaccgtttgaggtcggctccaacccatagatgaatttcttttttgtggccgtttctgggttacatgttttgggggaagggagtggaactgtccggttctttggcacacgtcaggttgctcttgatgcgcgacgtgcttgtatttgggtactgccgacaccaagcgtttcggcgattcctggaaaagagtgcctctcgctcgacgtttggttgttttctgcgtggtccgtcgtcgaccttcgttcgtccaaagacgccgtccggtttcatactcccccccgcacacatatcgaggccaattaaattgctaagggtgccgttgtcgtgcatctggcaggctcagaagtggcttatttgtcttttaattttgccgatgcacgcaaaaattgtcatttcttgaaagtttctcttttattgcgtacacaattcaacttttatgtaatttctgatggtctgttttacgtgtgcgtgtgtaaaacgtaactttggaagaatttttatgcacactgaacaaacgctcggtcctggggttgaaagtgctcggtgtgtgcatgagctaaagtgcaactgctttgttccgaaggttttctagtcgccgaaatgtaccattgtggaccttgttgcgagagaccttggtcttctgggggagctgctgtagcgtggcaagccactattttgggagcgacattgcagagaaaatcggcttttagaaaggcacctgcgcggcgagtggacgttttttcgtatatactgcgaaaaaaaaaaaaaaaaaaaaaaaaaaaaaaaaaaaaaaa

SEQ ID NO: 32 - protein, 425 aa, Ixodes ricinus
MPKGKRGPKAGGAARGGRCEASLAPSSSDEESNADTASVLSCASESRCGSDGTVGDPEAEEAVLHDDFEDKLKEAIDGASQKSAKGRLSCLEAIRKAFSTKYLYDFLMDRPSTVCDLVERGVRKGRGEEAALCATLGALACVQLGVGAEADALFDALRQPLCTLLLDGAQGPSPRARCATALGLCCFVVDSDNQLVLQPCMEVLWQVVGAKAGPGSPVLQAAALLAWGLLLSVAPVDRLLALTRTHLPRLQELLESPDLDLRIAAGEVIAVMYEGARDYDEDFEEPSESLCAQLRQLATDSQKFRAKKERRQQRSTFRDVYRAVREGASPDVSVKFGREVLELDTWSRKLQYDAFCQLLGSGMNLHLAVNELLRDIFELGQVLATEDHIISKITKFERHMVNMASCRARTKTRNRLRDKRADVVA

SEQ ID NO: 33 - DNA, 933 bp, Ixodes ricinus
  CDS (32)..(850)
gattgggaacctcctattcctcacttgaaacatggctggactccgctcctgcatcctcctggctcttgccactagtgccttcgccggctaccttcacggtggccttacccacggcgctgggtacggttacggtgtcggctacggttccggccttggctatggccttggctacggttccggccttggctatggacatgctgttggccttggacacggctttggctattctggtctgaccggctacagtgtggctgccccagctagctacgccgttgctgctccagccgtcagccgcaccgtttccacttaccacgctgctccagctgtggccacctacgccgctgctcctgtcgccacctatgctgttgctccagctgtcactagggtttcccccgttcgcgccgccccagctgtggccacgtacgccgccgctccagtcgccacctacgccgctgctccagctgtgaccagggtgtccaccattcacgctgccccggctgtggccaattacgccgtcgctccagtcgccacctatgccgctgctccagctgtgaccagggtgtccaccatccacgccgctccagccgtggctagctaccagacctaccacgctccagctgtcgccactgtggctcatgctccagctgtggccagctaccagacctaccacgctgccccagccgtggctacctacgcccatgccgctcccgtctacggctatggtgtcggtaccctcggatatggtgtcggccactacggctacggacacggtcttggcagctacggcctgaactacggttacggcctcggcacctacggtgactacaccacccttctccgcaagaagaagtaaatggcacatctcaagagagcccattggactgccatcgacattcttcttcaataaaagagcccgaagatggcattattttt

SEQ ID NO: 34 - protein, 273 aa, Ixodes ricinus
MAGLRSCILLALATSAFAGYLHGGLTHGAGYGYGVGYGSGLGYGLGYGSGLGYGHAVGLGHGFGYSGLTGYSVAAPASYAVAAPAVSRTVSTYHAAPAVATYAAAPVATYAVAPAVTRVSPVRAAPAVATYAAAPVATYAAAPAVTRVSTIHAAPAVANYAVAPVATYAAAPAVTRVSTIHAAPAVASYQTYHAPAVATVAHAPAVASYQTYHAAPAVATYAHAAPVYGYGVGTLGYGVGHYGYGHGLGSYGLNYGYGLGTYGDYTTLLRKKK

(19) United States
(12) Patent Application Publication
(10) Pub. No.: US 2003/0086937 A1
(43) Pub. Date: May 8, 2003

(54) Identification and molecular characterization of proteins, expressed in the Ixodes ricinus salivary glands

(76) Inventors: Edmond Godfroid, Brussels (BE); Alex Bollen, Itterbeek (BE); Gerard Leboulle, Brussels (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/165,605

(22) Filed: Jun. 7, 2002

Related U.S. Application Data
(63) Continuation-in-part of application No. 09/910,430, filed on Jul. 19, 2001.
Application No. 09/910,430 is a continuation-in-part of application No. PCT/BE00/00061, filed on Jun. 6, 2000.

(30) Foreign Application Priority Data
Jun. 9, 1999 (GB) GB9913425.6

(51) Int. Cl. A61K 39/00; C07H 21/04; C12N 9/64; C12P 21/02; C12N 5/06

(52) U.S. Cl. 424/185.1; 435/69.3; 435/226; 435/320.1; 435/348; 536/23.2

(57) ABSTRACT

The present invention relates to a new polynucleotide which encodes a polypeptide expressed in the salivary glands of ticks, more particularly the Ixodes ricinus arthropod tick, during the slow-feeding phase of the blood meal have. This polynucleotide and related polypeptide may be used in different constructions and for different applications which are also included in the present invention.

### CROSS REFERENCE TO RELATED APPLICATIONS

This application is a Continuation-in-Part of U.S. patent application Ser. No. 09/910,430 filed Jul. 19, 2001, which was a Continuation-in-Part of PCT/BE00/00061 filed on Jun. 6, 2000. The disclosures of each of the foregoing U.S. and PCT applications are hereby incorporated herein by reference in their entireties. PCT/BE00/00061 claims priority to GB9913425.6, filed Jun. 9, 1999, the disclosure of which is incorporated herein by reference in its entirety.

### FIELD OF THE INVENTION

The present invention is related to the molecular characterization of DNA sequences, which encode proteins expressed in the salivary glands of the Ixodes ricinus arthropod tick. These proteins are involved in the complex mechanism of interaction between this arthropod and its mammalian host. The invention relates to newly identified polynucleotides, polypeptides encoded by them and the use of such polynucleotides and polypeptides, and to their production.

### BACKGROUND OF THE INVENTION

Ticks are hematophagous arthropods that feed on a wide diversity of hosts. Unlike this group of arthropods, the Ixodid adult female ticks have the characteristics to ingest blood for an extended period of over 2 weeks.

Completion of the blood meal is dependent on the relationships of ticks with hosts species. Resistance to tick infestation implicates both innate and acquired immunity, and is characterized by reduced feeding, molting and mating capabilities that may lead to the death of the parasite. Acquired immunity of resistant hosts is mediated by a polarized Th1-type immune response, involving IFN-γ production and delayed type hypersensitivity reaction.

Some hosts are unable to counteract the tick infestation. Indeed, during their blood meal, ticks circumvent host defenses via pharmacologically active components secreted in their saliva. These factors can modulate both the innate and the acquired immunity of the host. In this way, the leukocyte responsiveness is modified during tick feeding. For example, cytokines production is modulated, inducing a polarised Th2 immune response.

Therefore, the complex tick-host molecular interaction can be considered as a balance between host defenses raised against the parasite and the tick evasion strategies, facilitating feeding for an extended period.

Although, there is extensive information about the effects of tick bioactive factors on host immune defenses, little is known about the mechanisms of their actions. However, it has been observed that a wide range of new proteins is expressed during the blood meal. Several of them might be essential for the completion of the tick feeding process.

### SUMMARY OF THE INVENTION

The present invention is related to a new isolated and purified polynucleotide obtained from tick salivary gland and presenting more than 75% identity with at least one nucleotide sequence selected from the group consisting of SEQ.ID.NO. 1, SEQ.ID.NO. 2, SEQ.ID.NO. 3, SEQ.ID.NO. 4, SEQ.ID.NO. 5, SEQ.ID.NO. 6, SEQ.ID.NO. 7, SEQ.ID.NO. 9, SEQ.ID.NO. 10, SEQ.ID.NO. 11, SEQ.ID.NO. 12, SEQ.ID.NO. 13, SEQ.ID.NO. 14, SEQ.ID.NO. 15, SEQ.ID.NO. 16, SEQ.ID.NO. 17, SEQ.ID.NO. 19, SEQ.ID.NO. 20, SEQ.ID.NO. 21, SEQ.ID.NO. 22, SEQ.ID.NO. 23, SEQ.ID.NO. 24, SEQ.ID.NO. 25, SEQ.ID.NO. 26, SEQ.ID.NO. 28, SEQ.ID.NO. 29, SEQ.ID.NO. 30, SEQ.ID.NO. 31, SEQ.ID.NO. 33 or a sequence complementary thereto, or a fragment thereof, as defined hereafter.

Preferably, the polynucleotide described above, which presents at least 80% identity with at least one of said nucleotide sequences, more preferably at least 90% identity, more preferably with at least 95% identity, and even at least about 98 to 99% identity.

Preferably, the polynucleotide of described above, which presents at least 99% identity with at least one of said nucleotide sequences.

The present invention is also related to a polypeptide encoded by the polynucleotide of the present invention or a biologically active fragment or portion thereof.

Said polypeptide may be modified by or linked to at least one substitution group, preferably selected from the group consisting of amide, acetyl, phosphoryl, and/or glycosyl groups.

Moreover, said polypeptide may take the form of a “mature” protein.

It may also be part of a larger protein or part of a fusion protein.

Preferably, the polypeptide of the present invention further includes at least one additional amino acid sequence which contains secretory or leader sequences, pro-sequences, sequences which help in purification such as multiple histidine residues, or additional sequences for stability during production of recombinant molecules.

Another object of the present invention concerns a variant of the polynucleotide or the polypeptide of the present invention, a precise definition of this term being given hereafter.

Preferably, said variant varies from the referent by conservative amino acid substitutions.

Preferably, at least one residue is substituted in said variant with another residue of similar characteristics.

Advantageously, the substitutions in said variant are among Ala, Val, Leu and Ile; among Ser and Thr, among the acidic residues Asp and Glu; among Asn and Gln; among the basic residues Lys and Arg; or among aromatic residues Phe and Tyr.

Preferably, in the variant of the present invention, several amino acids are substituted, deleted or added in any combination.

Preferably, 5-10, more preferably 1-5, more preferably 1-2 amino acids are substituted, deleted or added in any combination, in said variant.

Said variant may be a naturally occurring allelic variant of an Ixodes ricinus salivary gland polypeptide present in Ixodes ricinus salivary glands.

The present invention is also related to a recombinant vector comprising at least one element selected from the polynucleotide, the polypeptide, and the variant of the present invention or fragments thereof.

Another object of the present invention concerns a cell transfected by or comprising the recombinant vector according to the invention.

The present invention further includes an inhibitor directed against said polynucleotide, polypeptide, or variant.

Said inhibitor is preferably an antibody or an hypervariable portion thereof.

The present invention is also related to an hybridoma cell line expressing said inhibitor.

Another object of the present invention concerns a pharmaceutical composition comprising an adequate pharmaceutical carrier and an element selected from the group consisting of said polynucleotide, polypeptide, variant, vector, cell, inhibitor or a mixture thereof.

Preferably, said pharmaceutical composition presents anti-coagulant properties and advantageously contains at least one polynucleotide selected from the group consisting of SEQ.ID.NO. 7, SEQ.ID.NO. 17, and SEQ.ID.NO. 26, and fragments thereof or contains at least one polypeptide encoded by said polynucleotides or fragments thereof.

Preferably, the pharmaceutical composition presents immunomodulatory properties, and contains at least one polynucleotide selected from the group consisting of SEQ.ID.NO. 12, SEQ.ID.NO. 21, SEQ.ID.NO. 26, and SEQ.ID.NO. 31, and fragments thereof, or contains at least one polypeptide encoded by said polynucleotides or fragments thereof.

Another object of the invention is an immunological composition or vaccine for inducing an immunological response in a mammalian host to a tick salivary gland polypeptide which comprises at least one element of the group consisting of

a polynucleotide of tick salivary glands according to the invention;

a polypeptide of tick salivary glands according to the invention;

a variant according to the invention;

epitope-bearing fragments, analogs, outer-membrane vesicles or cells (attenuated or otherwise) of components a) or b) or c);

possibly a carrier.

The present invention is also related to a method for treating or preventing a disease affecting a mammal, said method comprising the step of administrating to said mammal a sufficient amount of the pharmaceutical composition or the immunological composition or vaccine according to the invention, in order to prevent or cure either the transmission of pathogenic agents by tick, especially by Ixodes ricinus, or the symptoms of diseases induced by tick or pathogenic agents transmitted by tick.

The present invention is also related to the use of the pharmaceutical composition or the immunological composition or vaccine according to the invention for the manufacture of a medicament in the treatment and/or prevention of diseases induced by tick or pathogenic agents transmitted by tick, especially by Ixodes ricinus.

Advantageously, said medicament may be used in transplantation, in rheumatology, but also in general treatment.

Finally, another object of the invention is a diagnostic kit for detecting a disease or susceptibility to a disease induced or transmitted by tick, especially Ixodes ricinus, which comprises:

at least one tick salivary gland polynucleotide of the invention, or a fragment thereof;

or at least one nucleotide sequence complementary to that of a);

or at least one tick salivary gland polypeptide, of the invention or a fragment thereof;

or at least one variant according to the invention or a fragment thereof

or an inhibitor of the invention;

or a phage displaying an antibody of the invention whereby a), b), c), d), e), f) may comprise a substantial component.

### BRIEF DESCRIPTION OF THE DRAWINGS

FIG. 1 presents results of RACE assay specific to SEQ.ID.NO. 17 and SEQ.ID.NO. 26. The reverse transcription step was carried out using 10 ng of mRNAs extracted from salivary gland of engorged ticks. The brightest bands represent the cDNA fragments corresponding to the 3′ end of the targeted mRNA. The amplified products were subjected to agarose gel electrophoresis followed by staining the DNA fragments by ethidium bromide. Arrows indicate the position of the expected amplified products.

FIG. 2 represents differential expression analysis of the 5 full-length selected cDNAs and 9 cDNA fragments isolated in the subtractive library. PCR assays were carried out using as DNA template cDNAs obtained from a reverse transcription procedure on mRNAs extracted from salivary glands either of engorged (E) or of unfed (UF) ticks. These RNA messengers were also used as template in reverse transcription assays. Ten microliter of both PCR and RT-PCR mixture were subjected to agarose gel electrophoresis and ethidium bromide staining for the detection of amplified DNA products. [++] strongly positive; [+] positive; [−] negative.

FIG. 3 represents a comparison of active sites of SEQ.ID.NO. 17 with its homologous sequences of different metallopeptidases (Factor X activating enzyme (FXA—accession n° A42972), Jararhagin (JAR—accession No. P30431), procollagen I—N proteinase (COL—accession No. HSAJ3125) and the mouse secretory protein containing thrombospondin motives (MSP—accession No. D67076). The consensus sequence of the zinc-binding motif is indicated below the alignment.

FIG. 4 represents confocal microscopy of female I. Ricinus salivary glands (A) Salivary glands of ticks fed during 5 days incubated with secondary antibody. Salivary glands of unfed ticks (B) and fed during 5 days (C) incubated with anti-SEQ.ID.NO. 17/MBP serum.

FIG. 5 represents the proliferation of cells from draining lymph nodes of mice pre-infested with I. ricinus nymphae. These cells were stimulated by different dilutions of culture media containing SEQ.ID.NO. 17/His or the negative control (NEG). The cells incorporation of tritiated thymidin was assessed on a scintillation counter.

### DEFINITIONS

“Putative anticoagulant, anti-complementary and immunomodulatory” cDNAs refer to polynucleotides having the nucleotide sequence described in the table, or allele variants thereof and/or their complements. These present homologies with anticoagulant, anti-complementary and immunomodulatory polynucleotides already existing in databases. These cDNAs belong to the Class I and Class II sequences (see table)

Some polypeptide or polynucleotide sequences present low or no homologies with already existing polypeptides or polynucleotides in databases. These belong to the Class III (see table).

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications include acetylation, acylation, ADP-ribosylation, amidation, covalent attachment of flavin, covalent attachment of a hem moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, PROTEINS—STRUCTURE AND MOLECULAR PROPERTIES, 2^{nd} Ed., T. E. Creighton, W. H. Freeman and Comany, New York, 1993 and Wolt, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182: 626-646 and Rattan et al, “Protein Synthesis: Posttranslational Modifications and Aging”, Ann NY Acad Sci (1992) 663:48-62.

“Polynucleotide” generally refers to any polyribonucleotide or polydeoxyribonucleotide, which may be unmodified RNA or DNA or modified RNA or DNA. “Polynucleotides” include, without limitation single- double-stranded DNA, DNA that is a mixture of single- double-stranded regions, single- double-stranded RNA, and RNA that is a mixture of single- double-stranded regions, hybrid molecules comprising DNA and RNA that may be single-stranded or, more typically, double-stranded or a mixture of single- double-stranded regions. In addition, “Polynucleotide” refers to triple-stranded regions comprising RNA or DNA or both RNA and DNA. The term “Polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “Polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more substitutions (preferably conservative), additions and deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide. For instance, they should have similar antigenic or immunogenic activities as the reference polypeptide. Antigenicity can be tested using standard immunoblot experiments, preferably using polyclonal sera against the reference polypeptide. The immunogenicity can be tested by measuring antibody responses (using polyclonal sera generated against the variant polypeptide) against purified reference polypeptide in a standard ELISA test. Preferably, a variant would retain all of the above biological activities.

“Identity” is a measure of the identity of nucleotide sequences or amino acid sequences. In general, the sequences are aligned so that the highest order match is obtained. “Identify” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds, Humana Press, New Jersey, 1994; SEQUENCE ANALYSIS IN MOLECULAR BIOLOGY, von Heijne, G., Academic Press, 1987; and SEQUENCE ANALYSIS PRIMER, Gribskov, M. and Devereux, J., eds, M Stockton Press, New York, 1991). While there exist a number of methods to measure identity between two polynucleotide or polypeptide sequences, the term “identity” is well known to skilled artisans (Carillo, H., and Lipton, D., SIAM J Applied Math (1998) 48:1073). Methods commonly employed to determine identity or similarity between two sequences include, but are not limited to those disclosed in Guide to Huge Computers, Martin J. Bishop, ed., Academic Press, San Diego, 1994, and Carillo, H., and Lipton, D., SIAM J Applied Math (1988) 48:1073. Methods to determine identity and similarity are codified in computer programs. Preferred computer program methods to determine identity and similarity between two sequences include, but are not limited to, GCG program package (Devereux, J., et al., J Molec Biol (1990) 215:403). Most preferably, the program used to determine identity levels was the GAP program, as was used in the Examples hereafter.

As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

Fragments of I. ricinus salivary gland polypeptides are also included in the present invention. A fragment is a polypeptide having an amino acid sequence that is the same as a part, but not all, of the amino acid sequence of the aforementioned I. ricinus salivary gland polypeptides. As with I. ricinus salivary gland polypeptides, fragment may be “free-standing” or comprised within a larger polypeptide of which they form a part or region, most preferably as a single continuous region. Representative examples of polypeptide fragments of the invention, include, for example, fragments from about amino acid number 1-20, 21-40, 41-60, 61-80, 81-100, and 101 to the end of the polypeptide. In this context “about” includes the particularly recited ranges larger or smaller by several, 5, 4, 3, 2 or 1 amino acid at either extreme or at both extremes.

Preferred fragments include, for example, truncated polypeptides having the amino acid sequence of the I. ricinus salivary gland polypeptides, except for deletion of a continuous series of residues that includes the amino terminus, or a continuous series of residues that includes the carboxyl terminus and/or transmembrane region or deletion of two continuous series of residues, one including the amino terminus and one including the carboxyl terminus. Also preferred are fragments characterised by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate I. ricinus salivary gland protein activity, including those with a similar activity or an improved activity, or with a decreased undesirable activity. Also included are those that are antigenic or immunogenic in an animal or in a human.

### EXAMPLES

### Example 1

### Characterization of the Induced Genes

Genes are induced in the salivary glands of Ixodes ricinus during the slow-feeding phase of the blood meal. The cloning of these genes was carried out by setting up two complementary DNA (CDNA) libraries. The first one is a subtractive library based on the methodology described by Lisitsyn et al. (Science 259, 946-951,1993) and improved by Diatchenko et al. (Proc. Natl. Acad. Sci. USA 93, 6025-6030, 1996). This library cloned selectively induced mRNA during the tick feeding phase. The second library is a full-length CDNA library, which was constructed by using the basic property of mRNAs (presence of a polyA tail in its 3′end and a cap structure in its 5′ end). This cDNA library permitted the cloning of full-length cDNAs, corresponding to some incomplete CDNA sequences identified in the subtractive cDNA library.

The subtractive library was set up by subtracting uninduced-cDNAs (synthetized from mRNAs equally expressed in the salivary glands of both unfed and engorged ticks) from induced-cDNAs (synthesised from mRNAs differentially expressed in the salivary gland at the end of the slow-feeding phase). The induced-cDNAs was digested by a restriction enzyme, divided into two aliquots, and distinctively modified by the addition of specific adapters. As for the induced-cDNAs, the uninduced cDNAs was also digested by the same restriction enzyme and then mixed in excess to each aliquot of modified induced-cDNA. Each mixture of uninduced-/induced-cDNAs was subjected to a denaturation step, immediately followed by an hybridisation step, leading to a capture of homologous induced-cDNAs by the uninduced-cDNA. Each mixture was then mixed together and subjected again to a new denaturation/hybridisation cycle. Among the hybridised cDNA molecules, the final mixture comprises induced-cDNAs with different adapters at their 5′ and 3′ end. These relevant cDNAs were amplified by polymerase chain reaction (PCR), using primers specific to each adapter located at each end of the cDNA molecules. The PCR products were then ligated into the PCRII™ vector by A-T cloning and cloned in an TOP-10 E. Coli strain. The heterogeneity of this subtractive library was evaluated by sequencing 96 randomly chosen recombinant clones. The “induced” property of these CDNA sequences was checked by reverse transcription-PCR (RT-PCR) on mRNA extracted from salivary glands of engorged and unfed ticks. Finally, the full-length induced-cDNA was obtained by screening the full-length cDNA library using, as a probe, some incomplete induced-cDNAs isolated from the subtractive library. These full-length induced DNA molecules were sequenced and compared to known polypeptide and polynucleotide sequences existing in the EMBL/GenBank databases.

The full-length cDNA library was set up by using the strategy developed in the “CapFinder PCR cDNA Library Construction Kit” (Clontech). This library construction kit utilises the unique CapSwitch™ oligonucleotide (patent pending) in the first-strand synthesis, followed by a long-distance PCR amplification to generate high yields of full-length, double-stranded cDNAs. All commonly used cDNA synthesis methods rely on the ability of reverse transcriptase to transcribe mRNA into single stranded DNA in the first-strand reaction. However, because the reverse transcriptase cannot always transcribe the entire mRNA sequence, the 5′ ends of genes tend to be under-represented in cDNA population. This is particularly true for long mRNAs, especially if the first-strand synthesis is primed with oligo(dT) primers only, or if the mRNA has a persistent secondary structure. Furthermore, the use of T4 DNA polymerase to generate blunt cDNA ends after second-strand synthesis commonly results in heterogeneous 5′ ends that are 5-30 nucleotides shorter than the original mRNA (D'Alessio, 1988). In the CapFinder cDNA synthesis method, a modified oligo(dT) primer is used to prime the first-strand reaction, and the CapSwitch oligonucleotide acts as a short, extended template at the 5′ end for the reverse transcriptase. When the reverse transcriptase reaches the 5′ end of the mRNA, the enzyme switches templates and continues replicating to the end of the CapSwitch oligonucleotide. This switching in most cases occurs at the 7-methylguanosine cap structure, which is present at the 5′ end of all eukaryotic mRNAs (Furuichi & Miura, 1975). The resulting full-length single stranded cDNA contains the complete 5′ end of the mRNA as well as the sequence complementary to the CapSwitch oligonucleotide, which then serves as a universal PCR priming site (CapSwitch anchor) in the subsequent amplification. The CapSwitch-anchored single stranded cDNA is used directly (without an intervening purification step) for PCR. Only those oligo(dT)-primed single stranded cDNAs having a CapSwitch anchor sequence at the 5′ end can serve as templates and be exponentially amplified using the 3′ and 5′ PCR primers. In most cases, incomplete cDNAs and cDNA transcribed from poly-A RNA will not be recognized by the CapSwitch anchor and therefore will not be amplified.

At the end of these reactions, the full-length cDNA PCR products was ligated into the pCRII cloning vector (Invitrogen) and used for the transformation of XL2 E. coli strain. The full-length cDNA library was then screened by using, as a probe, the incomplete induced-cDNAs isolated from the subtractive library.

Ninety-six clones of subtractive library were randomly sequenced, and their DNA and amino acid translated sequences were compared to DNA and protein present in databases. Among these, 27 distinct family sequences were identified, and 3 of them were selected for further characterization of their corresponding full-length mRNA sequence. These 3 sequences matched the sequence of i) the human tissue factor pathway inhibitor (TFPI), ii) the human thrombin inhibitor gene, and iii) a snake venom zinc-dependent metalloprotease protein. These genes encode proteins that could be involved in the inhibition of the blood coagulation. The other 24 family sequences presented low or no homologies with polynucleotide and polypeptide sequences existing in databases. Screening of the full-length cDNA library using oligonucleotide probes specific to the 3 previously selected subtractive clones lead to the recovery of the corresponding full-length cDNAs. Random screening of this library led to the selection of 2 other clones. One is closely homologous to an interferon-like protein, whereas the other shows homologies to the Streptococcus equi M protein, an anti-complement protein.

These polypeptides expressed by I. ricinus salivary glands include the polypeptides encoded by the cDNAs defined in the tables, and polypeptides comprising the amino acid sequences which have at least 75% identity to that encoded by the cDNAs defined in the tables over their complete length, and preferable at least 80% identity, and more preferably at least 90% identity. Those with about 95-99% are highly preferred.

The I. ricinus salivary gland polypeptides may be in the form of the “mature” protein or may be a part of a larger protein such as a fusion protein. It may be advantageous to include an additional amino acid sequence, which contains secretory or leader sequences, pro-sequences, sequences which help in purification such as multiple histidine residues, or an additional sequence for stability during recombinant production.

Preferably, all of these polypeptide fragments retain parts of the biological activity (for instance antigenic or immunogenic) of the I. ricinus salivary gland polypeptides, including antigenic activity. Variants of the defined sequence and fragments also form part of the present invention. Preferred variants are those that vary from the referents by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination. Most preferred variants are naturally occurring allelic variants of the I. ricinus salivary gland polypeptide present in I. ricinus salivary glands.

The I. ricinus salivary gland polypeptides of the invention can be prepared in any suitable manner. Such polypeptides include isolated naturally occurring polypeptides, recombinant polypeptides, synthetic polypeptides, or polypeptides produced by a combination of these methods. Means for preparing such polypeptides are well understood in the art.

The I. ricinus salivary gland cDNAs (polynucleotides) include isolated polynucleotides which encode I. ricinus salivary gland polypeptides and fragments thereof, and polynucleotides closely related thereto. More specifically, I. ricinus salivary gland cDNAs of the invention include a polynucleotide comprising the nucleotide sequence of cDNAs defined in the table, encoding a I. ricinus salivary gland polypeptide. The I. ricinus salivary gland cDNAs further include a polynucleotide sequence that has at least 75% identity over its entire length to a nucleotide sequence encoding the I. ricinus salivary gland polypeptide encoded by the cDNAs defined in the tables, and a polynucleotide comprising a nucleotide sequence that is at least 75% identical to that of the cDNAs defined in the tables, in this regard, polynucleotides at least 80% identical are particularly preferred, and those with at least 90% are especially preferred. Furthermore, those with at least 95% are highly preferred and those with at least 98-99% are most highly preferred, with at least 99% being the most preferred. Also included under I. ricinus salivary gland cDNAs is a nucleotide sequence, which has sufficient identity to a nucleotide sequence of a cDNA defined in the tables to hybridise under conditions usable for amplification or for use as a probe or marker. The invention also provides polynucleotides which are complementary to such I. ricinus salivary gland cDNAs.

These nucleotide sequences defined in the tables as a result of the redundancy (degeneracy) of the genetic code may also encode the polypeptides encoded by the genes defined in the tables.

When the polynucleotides of the invention are used for the production of an I. ricinus salivary gland recombinant polypeptide, the polynucleotide may include the coding sequence for the mature polypeptide or a fragment thereof, by itself, the coding sequence for the mature polypeptide or fragment in reading frame with other coding sequences, such as those encoding a leader or secretory sequence, a pre-, or pro-or preproprotein sequence, or other fusion peptide portions. For example, a marker sequence, which facilitates purification of the fused polypeptide can be encoded. Preferably, the marker sequence is a hexa-histidine peptide, as provided in the pQE vector (Qiagen, Inc.) and described in Gentz et al, Proc Natl Acad Sci USA (1989) 86:821-824, or is an HA tag, or is glutathione-s-transferase. The polynucleotide may also contain non-coding 5′ and 3′ sequences, such as transcribed, non-translated sequences, splicing and polyadenylation signals, ribosome binding sites and sequences that stabilize mRNA.

Further preferred embodiments are polynucleotides encoding I. ricinus salivary gland protein variants comprising the amino acid sequence of the I. ricinus salivary gland polypeptide encoded by the cDNAs defined by the table respectively in which several, 10-25, 5-10, 1-5, 1-3, 1-2 or 1 amino acid residues are substituted, deleted or added, in any combination. Most preferred variant polynucleotides are those naturally occurring I. ricinus sequences that encode allelic variants of the I. ricinus salivary gland proteins in I. ricinus.

The present invention further relates to polynucleotides that hybridise preferably stringent conditions to the herein above-described sequences. As herein used, the term “stringent conditions” means hybridisation will occur only if there is at least 80%, and preferably at least 90%, and more preferably at least 95%, yet even more preferably 97-99% identity between the sequences.

Polynucleotides of the invention, which are identical or sufficiently identical to a nucleotide sequence of any gene defined in the table or a fragment thereof, may be used as hybridisation probes for CDNA clones encoding I. ricinus salivary gland polypeptides respectively and to isolate CDNA clones of other genes (including cDNAs encoding homologs and orthologs from species other than I. ricinus) that have a high sequence similarity to the I. ricinus salivary gland cDNAs. Such hybridisation techniques are known to those of skill in the art. Typically these nucleotide sequences are 80% identical, preferably 90% identical, more preferably 95% identical to that of the referent. The probes generally comprise at least 15 nucleotides, preferably, at least 30 nucleotides or at least 50 nucleotides. Particularly preferred probes range between 30 and 50 nucleotides. In one embodiment, to obtain a polynucleotide encoding I. ricinus salivary gland polypeptide, including homologues and orthologues from species other than I. ricinus, comprises the steps of screening an appropriate library under stringent hybridisation conditions with a labelled probe having a nucleotide sequence contained in one of the gene sequences defined by the table, or a fragment thereof, and isolating full-length cDNA clones containing said polynucleotide sequence. Thus in another aspect, I. ricinus salivary gland polynucleotides of the present invention further include a nucleotide sequence comprising a nucleotide sequence that hybridise under stringent condition to a nucleotide sequence having a nucleotide sequence contained in the cDNAs defined in the tables or a fragment thereof. Also included with I. ricinus salivary gland polypeptides are polypeptides comprising amino acid sequences encoded by nucleotide sequences obtained by the above hybridisation conditions (conditions under overnight incubation at 42° C. in a solution comprising: 50% formamide, 5×SSC (150mM NaCl, 15mM trisodium citrate), 50mM sodium phosphate (pH 7.6), 5× Denhardt's solution, 10% dextran sulfate, and 20 microgram/ml denatured, sheared salmon sperm DNA, followed by washing the filters in 0.1×SSC at about 65° C.).

The polynucleotides and polypeptides of the present invention may be employed as research reagents and materials for the development of treatments and diagnostics tools specific to animal and human disease.

This invention also relates to the use of I. ricinus salivary gland polypeptides, or I. ricinus salivary gland polynucleotides, for use as diagnostic reagents.

Materials for diagnosis may be obtained from a subject's cells, such as from blood, urine, saliva, tissue biopsy.

Thus in another aspect, the present invention relates to a diagnostic kit for a disease or susceptibility to a disease which comprises:

(a) an I. ricinus salivary gland polynucleotide, preferably the nucleotide sequence of one of the gene sequences defined by the table, or a fragment thereof;

(b) a nucleotide sequence complementary to that of(a);

(c) an I. ricinus salivary gland polypeptide, preferably the polypeptide encoded by one of the gene sequences defined in the table, or a fragment thereof;

(d) an antibody to an I. ricinus salivary gland polypeptide, preferably to the polypeptide encoded by one of the gene sequences defined in the table; or

(e) a phage displaying an antibody to an I. ricinus salivary gland polypeptide, preferably to the polypeptide encoded by one of the cDNAs sequences defined in the table.

It will be appreciated that in any such kit, (a), (b), (c), (d) or (e) may comprise a substantial component.

Another aspect of the invention relates to a method for inducing an immunological response in a mammal which comprises inoculating the mammal with I. ricinus salivary gland polypeptide or epitope-bearing fragments, analogues, outer-membrane vesicles or cells (attenuated or otherwise), adequate to produce antibody and/or T cell immune response to protect said animal from bacteria and viruses which could be transmitted during the blood meal of I. ricinus and related species. In particular the invention relates to the use of I. ricinus salivary gland polypeptides encoded by the cDNAs defined in the tables. Yet another aspect of the invention relates to a method of inducing immunological response in a mammal which comprises, delivering I. ricinus salivary gland polypeptide via a recombinant vector directing expression of I. ricinus salivary gland polynucleotide in vivo in order to induce such an immunological response to produce antibody to protect said animal from diseases transmitted by I. ricinus ticks or other related species (Lyme disease, tick encephalitis virus disease, . . . ).

A further aspect of the invention relates to an immunological composition or vaccine formulation which, when introduced into a mammalian host, induces an immunological response in that mammal to a I. ricinus salivary gland polypeptide wherein the composition comprises a I. ricinus salivary gland CDNA, or I. ricinus salivary gland polypeptide or epitope-bearing fragments, analogs, outer-membrane vesicles or cells (attenuated or otherwise). The vaccine formulation may further comprise a suitable carrier. The I. ricinus salivary gland polypeptide vaccine composition is preferably administered orally or parenterally (including subcutaneous, intramuscular, intravenous, intradermal injection). Formulations suitable for parenteral administration include aqueous and non-aqueous sterile injection solutions which may contain anti-oxidants, buffers, bacteriostats and solutes which render the formulation iotonic with the blood of the recipient; and aqueous and non-aqueous sterile suspensions which may include suspending agents or thickening agents. The formulations may be presented in unit-dose or multi-dose containers, for example; sealed ampoules and vials and may be stored in a freeze-dried condition requiring only the addition of the sterile liquid carrier immediately prior to use. The vaccine formulation may also include adjuvant systems for enhancing the immunogenicity to the formulation, such as oil-in water systems and other systems known in the art. The dosage will depend on the specific activity of the vaccine and can be readily determined by routine experimentation.

Yet another aspect relates to an immunological/vaccine formulation which comprises the polynucleotide of the invention. Such techniques are known in the art, see for example Wolff et al, Sciences, (1990) 247: 1465-8.

Another aspect of the invention related to the use of these I. ricinus salivary gland polypeptides as therapeutic agents. In considering the particular potential therapeutic areas for such products, the fields covered by these products are: haematology (particularly coagulation clinics), transplantation (for immunosuppression control), rheumatology (for anti-inflammatories), and general treatment (for specific or improved anaesthetics).

TABLE 1
Sequences identified in the subtractive and the cDNA full-length libraries

| | Motifs | Similar sequences in Databases | Score | Class |
|---|---|---|---|---|
| Seq. 1 | | No significative identity | | III |
| Seq. 2 | | No significative identity | | III |
| Seq. 3 | | No significative identity | | III |
| Seq. 4 | | No significative identity | | III |
| Seq. 5 | Prokariotic mbre lipoprotein lipid attachment site | No significative identity | | III |
| Seq. 6 | | R. melioti Nitrogen fixation (fixF) | 0.00089 | III |
| | | Human Apolipoprotein B-100 | 0.0045 | III |
| | | Hu mRNA for cAMP response element (CRE-BP1) binding prot | 0.057 | III |
| Seq. 7 | Kunitz family of serine protease inhibitor | Human BAC clone GS345D13 | 4.7¹³ | I |
| | | H. sap Tissue factor Pathway Inhibitor | 4⁻¹² | I |
| Seq. 8 | | No significative identity | | |
| Seq. 9 | Prokariotic membrane lipoprotein lipid attachment site | No significative identity | | III |
| Seq. 10 | | Pea mRNA for GTP binding protection | 0.48 | III |
| Seq. 11 | | No significative identity | | III |
| Seq. 12 | | IL-11 R-Beta gene | 0.18 | II |
| Seq. 13 | | No significative identity | | III |
| Seq. 14 | | C. gloeosporioides cutinase gene | 0.082 | III |
| Seq. 15 | | No significative identity | | III |
| Seq. 16 | | Mouse mRNA for secretory protection cont. thrombospondin | 0.014 | III |
| | | motifs | | |
| Seq. 17 | Zinc dependent metallopeptidase family | B. jararaca mRNA for jararhagin | 1.1⁻⁵ | I |
| | | Agkistrodon contortrix metalloproteinase precursor | 3.9⁻⁵ | I |
| Seq. 19 | | O. anes gene for ovine Interferon-alpha | 0.7 | II |
| | | Interferon-omega 45 | 0.88 | II |
| | | Interferon-omega 20 | 0.89 | II |
| | | RCPT PGE2 | 0.85 | III |
| | | PGE Rcpt EP2 | 0.85 | III |
| Seq. 20 | | No significative identity | | III |
| Seq. 21 | | IgG1L chain directed against human IL2 rcpt Tac protein | 0.19 | II |
| | | Var region of light chain of MAK447/179 | 0.2 | II |
| Seq. 22 | | No significative identity | | III |
| Seq. 23 | | No significative identity | | III |
| Seq. 24 | | Mus Musculus neuroactin | 0.42 | III |
| Seq. 26 | | H. sapiens thrombin inhibitor | 2.1⁻¹² | I |
| | | Cycloplasmic antiproteinase 38 kDa intracellular serine | 2.3⁻¹² | I |
| | | protection. | | |
| Seq. 28 | | No significative identity | | III |
| Seq. 29 | | No significative identity | | III |
| Seq. 30 | | Mus musculus transcription factor ELF3 (fasta) | 0.053 | III |
| Seq. 3128 | | Homo sapiens putative leukocyte interferon-related protein | 1.70E−22 | II |
| | | (SM15) mRNA | | |
| Seq. 33 | | R. norvegicus mRNA for common antigen-related protein | 4.80E−09 | II |

(SEQ. ID. NO. 26 (Iris) homology with H. sapiens thrombin inhibitor 2.1-12, class I
Class I: putative anticoagulant homologs;
Class II: putative immunomodulatory homologs;
Class III: low or no homologies found in the databases).

TABLE 2
Biological characteristics of the selected clones

| | Full-length sequences | Fasta/Blastp | ORF | | Signal peptide | | Nucleotide in |
|---|---|---|---|---|---|---|---|
| Clone | similarly to databases | Scores^{a} | (aa) | Motifs | scores^{b} | Sp length/Prob. | position −3^{c} |
| Seq31 | Homo sapiens putative interferon-related | 1,8.10⁻³⁶/1.10⁻⁷¹ | 426 | | D | 48aa/8,4.10⁻¹ | G |
| | gene (SKMc15) [U09585] | | | | 5,4/F^{e} | | |
| Seq33 | R. norvegicus leukocyte common antigen | 7,8.10⁻¹¹/N | 274 | | 10,2/S | 18aa/7,4.10⁻³ | A |
| | (LAR) mRNA [X83546] | | | | | | |
| Seq17 | Mouse mRNA for secretory protein | 0,002/6.10⁻⁷ | 489 | Metallopeptidase | 7,9/S | 19aa/7,4.10⁻⁴ | G |
| | containing thrombospondin motives | | | | | | |
| | [D67076] | | | | | | |
| Seq26 | Pig leukocyte elastase inhibitor mRNA | 0/7.10⁻⁶⁷ | 378 | Serpin | 8,5/S | 51aa/3,28.10⁻³ | A |
| | [P80229] | | | | | | |
| Seq7 | Human Tissue Factor Pathway Inhibitor | 4,8.10⁻¹²/2.10⁻⁵ | 87 | Kunitz | 6,5/S | 19aa:1,8.10⁻⁴ | G |
| | [P48307] | | | | | | |

^{a}No score (N)
^{b}Succeeded (S) and Failed (F)
^{c}Guanine (G) and Adenine (A)
^{d}von Heijne analysis
^{e}McGeoch analysis

### Example 2

### Construction of a Representational Difference Analysis (RDA) Subtractive Library

The salivary glands of 5 day engorged or unfed free of pathogen I. ricinus female adult ticks were used in this work.

When removed, these glands were immediately frozen in liquid nitrogen and stored at −80° C. To extract RNA messengers (mRNA), the salivary glands were crushed in liquid nitrogen using a mortar and a pestle. The mRNAs were purified by using an oligo-dT cellulose (Fast Track 2.0 kit, Invitrogen, Groningen, The Netherlands). Two micrograms of mRNAs were extracted from 200 salivary glands of fed ticks, and 1.5 μg of mRNAs were also extracted from 1,000 salivary glands of unfed ticks.

All procedures were performed as described by Hubank and Schatz (1994). Double-stranded cDNAs were synthesised using the Superscript Choice System (Life Technologies, Rockville, Md. USA). The cDNAs were digested with DpnII restriction enzyme, ligated to R-linkers, amplified with R-24 primers (Hubank and Schatz, 1994), and finally digested again with the same enzyme to generate a “tester” pool consisting of cDNAs from salivary glands of fed ticks and a “driver” pool consisting of cDNAs from salivary glands of unfed ticks. The first round of the subtractive hybridisation process used a tester/driver ratio of 1:100. The second and third rounds utilised a ratio of 1:400 and 1:200,000, respectively. After three cycles of subtraction and amplification, the DpnII-digested differential products were subdivided according to size into 4 different fractions on a 1.7% electrophoresis agarose gel, and subcloned the BamHI site of the pTZ19r cloning vector. The ligated product was used to transform TOP-10 E. coli competent cells (Invitrogen, Groningen, The Nederlands). Nine thousand six hundred clones of this subtractive library were randomly selected, and individually put in 96-well microplates and stored at −80° C. This subtractive library was analysed by sequencing 89 randomly chosen clones, using M13 forward and reverse primers specific to a region located in the pT19r cloning vector. The DNA sequences of these 89 clones were compared, and 27 distinct family sequences were identified. Homology of these sequences to sequences existing in databases is presented in Table 1.

The subtractive sequences 1 to 27 are presented in the sequence-listing file (except for sequences 7, 17 and 26 whose complete mRNA sequences are presented; see also Example 2). Three sequences (SEQ.ID.NO. 7, 17 and 26) were selected for further characterization of their corresponding full-length mRNA sequence. These 3 sequences matched the sequence of i) the human tissue factor pathway inhibitor (TFPI), ii) a snake venom zinc dependent metallopeptidase protein, and iii) the human thrombin inhibitor protein, corresponding to SEQ.ID.NO. 7, 17 and 26, respectively. These genes encode proteins which could be involved in the inhibition of the blood coagulation or in the modulation of the host immune response.

### Example 3

### Construction of the Full Length cDNA Library and Recovery of Full Length cDNAs Sequences by Screening of this Full Lenth cDNA Library

This library was set up using mRNAs extracted from salivary glands of engorged ticks. The mRNAs (80 ng) were subjected to reverse transcription using a degenerated oligo-dT primer (5′A(T)30VN-3′), the Smart™ oligonucleotide (Clontech, Palo Alto, USA), and the Superscript II reverse transcriptase (Life Technologies, Rockville, Md., USA). The single strand cDNA mixture was used as template in a hot start PCR assay including the LA Taq polymerase (Takara, Shiga, Japan), the modified oligo-dT primer and a 3′-Smart primer specific to a region located at the 5′ end of the Smart™ oligonucleotide. The PCR protocol applied was: 1 min at 95° C., followed by 25 sec at 95° C./5 min at 68° C., 25 times; and 10 min at 72° C. The amplified double stranded cDNA mixture was purified with a Centricon 30 concentrator (Millipore, Bedford, USA). The cDNAs were divided into 4 fractions ranging from 0.3 to 0.6 kb, 0.6 to 1 kb, 1 kb to 2 kb, and 2 kb to 4 kb on a 0,8% high grade agarose electrophoresis gel. Each fraction was recovered separately by using the Qiaex II extraction kit (Qiagen, Hilden, Germany). The 4 fractions were ligated individually into the pCRII cloning vector included in the TOPO cloning kit (Invitrogen, Groningen, The Netherlands). The ligated fractions were then used to transform XL2-Blue ultracompetent E. coli cells (Stratagene, Heidelburg, Germany). The resulted recombinant clones were stored individually in microplates at −80° C. Ten clones were randomly chosen for partial or complete sequencing. As a result of this procedure, 2 cDNA sequences (SEQ.ID.NO. 31 and SEQ.ID.NO. 33, see Table 1) were selected for their homology to sequence databases. One is closely homologous to an interferon-related protein (SEQ.ID.NO. 31), whereas the other shows homologies to the Rattus norvegicus leukocyte common antigen-related protein (SEQ.ID.NO. 33).

The 4 different fractions of the full-length CDNA library were screened with radiolabelled oligonucleotide probes specific to selected clones identified in the subtractive cDNA library. The labelling of these oligo probes was performed as described in “Current Protocols in Molecular Biology” (Ausubel et al, 1995, J. Wiley and sons, Eds). These 4 different fractions were then plated on nitrocellulose membranes and grown overnight at 37° C. These membranes were denatured in NaOH 0.2 M/NaCl 1.5M, neutralised in Tris 0.5M pH 7.5-NaCl 1.5M and fixed in 2×SSC (NaCl 0.3 M/Citric Acid Trisodium di-hydrated 0.03 M). The membranes were heated for 90 min. at 80° C., incubated in a pre-hybridisation solution (SSC 6×, Denhardt's 10×, SDS 0,1%) at 55° C. for 90 min., and finally put overnight in a preheated hybridisation solution containing a specific radiolabelled oligonucleotide probe at 55° C. The hybridised membranes were washed 3 times in a SSC 6× solution at 55° C. for 10 min, dried and exposed on Kodak X-OMAT film overnight at −80° C.

The full-length cDNA library was also analysed by sequencing a set of clones. The resulted DNA sequences were compared to EMBL/GenBank databases and were used to set up oligonucleotide probes to recover other corresponding clones. In this way, the complete consensus mRNA sequence of the SEQ.ID.NO. 28 and 29 was confirmed by the recovery of two other clones corresponding to these sequences. Only one full-length cDNA clone corresponding to the subtractive clone 17 was isolated. Therefore, to identify the complete sequence of the SEQ.ID.NO. 17 and SEQ.ID.NO. 26, the Rapid Amplification of cDNA Ends (RACE) method was applied.

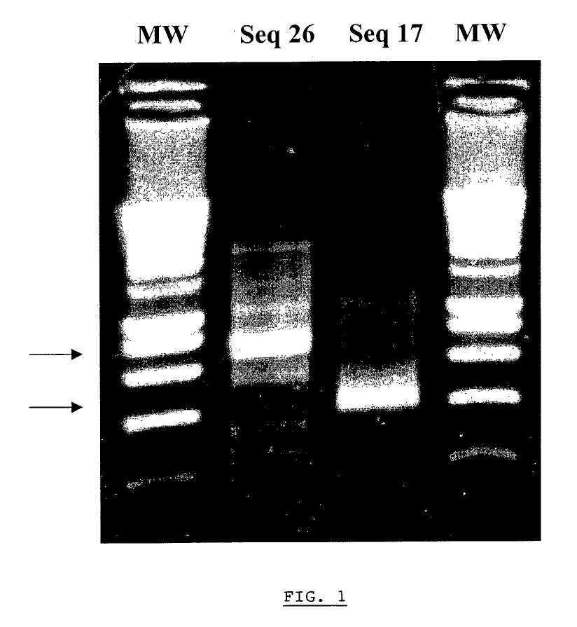

The RACE methodology was performed as described by Frohman et al. (1995). The reverse transcription step was carried out using 10 ng of mRNAs extracted from salivary glands of engorged ticks and the Thermoscript Reverse transcriptase (Life technologies, Rockville, Md., USA). All gene specific primers (GSP) had an 18 base length with a 61% G/C ratio. The amplified products were subjected to an agarose gel electrophoresis and recovered by using an isotachophorese procedure. The cDNAs were cloned into the pCRII-TOPO cloning vector (Invitrogen, Groningen, The Netherlands). To identify the consensus cDNA sequence, different clones were sequenced, and their sequence was compared to their known corresponding sequence. Therefore, the complete cDNA sequences of the clones 17 and 26 isolated in the subtractive library were obtained by this RACE procedure (FIG. 1).

### Example 4

### Analysis of the Full Sequences of 5 Selected Clones

The sequences of selected clones (SEQ.ID.NO. 7, 17, 26, 31 and 33) allowed identification of the open reading frames, from which the amino sequences were deduced. These potential translation products have a size between 87 and 489 amino acids (see table 2). In order to evaluate, in silico, their respective properties, the amino acid sequences and the nucleotide sequences of said 5 open frames were compared with the databases using the tFasta and Blastp algorithms.

These comparisons show that SEQ.ID.NO. 7 is highly homologous to the human Tissue Factor Pathway Inhibitor (TFPI). TFPI is an inhibitor of serine proteases having 3 tandemly arranged Kunitz-type-protease-inhibitor (KPI) domains. Each of these units or motifs has a particular affinity for different types of proteases. The first and second KPI domains are responsible for the respective inhibition of VIla and Xa coagulation factors. The third KPI domain apparently has no inhibitory activity. It should be noted that the corresponding polypeptide sequence of SEQ.ID.NO. 7 cDNA clone is homologous to the region of the first KPI domain of TFPI and that the KPI is perfectly kept therein. This similarity suggests that the SEQ.ID.NO. 7 protein is a potential factor VIla inhibitor.

The amino sequence deduced from the SEQ.ID.NO. 28 clone has a great homology with 3 database sequences, namely: mouse TIS7 protein, rat PC4 protein and human SKMc15 protein. These 3 proteins are described as putative interferon type factors. They possess very well conserved regions of the B2 interferon protein. Therefore, it is proposed that the SEQ.ID.NO. 3 1 protein has advantageous immunomodulatory properties.

Sequences SEQ.ID.NO. 17 and SEQ.ID.NO. 26 were compared with databases showing homology with the Gloydius halys (sub-order of ophidians) M12b metallopeptidase and the porcine elastase inhibitor belonging to the super-family of the serine protease inhibitors (Serpin), respectively. The amino sequences of these 2 clones also have specific motifs of said families. It is proposed that said proteins have advantageous anticoagulant and immuno-modulatory properties.

Finally, the SEQ.ID.NO. 33 clone has a weak homology with the R. norvegicus leukocyte common antigen (LAR) that is an adhesion molecule. It is thus possible that the SEQ.ID.NO. 33 protein has immunomodulatory properties related to those expressed by the LAR protein.

Due to their potential properties, most of the proteins examined are expected to be secreted in the tick saliva during the blood meal. Accordingly, tests were made for finding the presence of a signal peptide at the beginning of the deduced amino sequences. By the McGeoch method (Virus Res 3: 271-286, 1985), signal peptide sequences were detected for the SEQ.ID.NO. 7, SEQ.ID.NO. 17, SEQ.ID.NO. 26 and SEQ.ID.NO. 33 deduced amino sequences. It seems that said proteins are secreted in the tick salivary gland. Furthermore, the presence of a Kozak consensus sequence was observed upstream of the coding sequences of all examined clones. This indicates that their mRNAs potentially could be translated to proteins.

### Example 5

### Evaluation of the Differential Expression of the cDNA Clones Isolated in the Subtractive and Full-Length cDNA Libraries

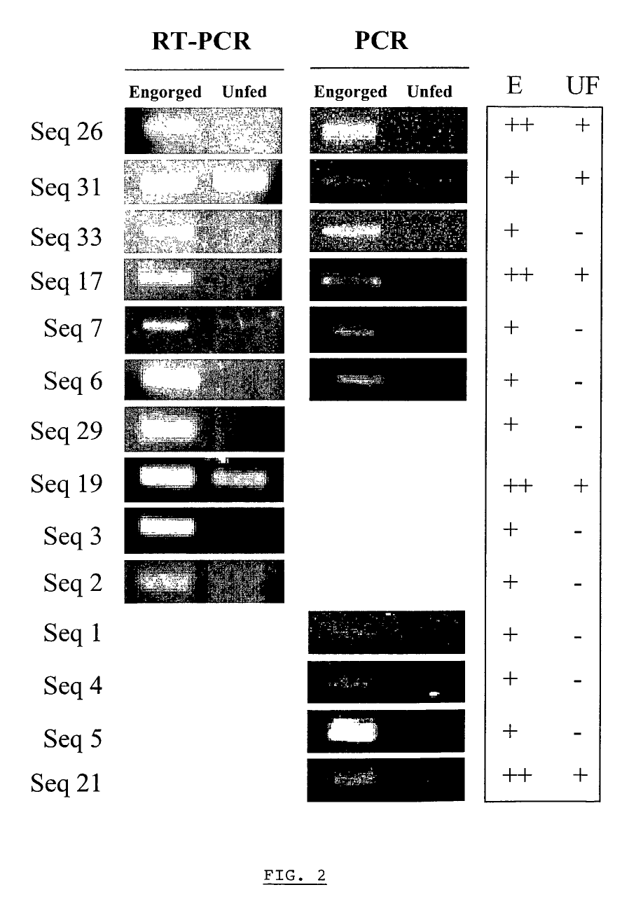

The differential expression of the mRNAs corresponding to the 5 full-length selected clones (SEQ.ID.NO. 7, SEQ.ID.NO. 17, SEQ.ID.NO. 26, SEQ.ID.NO. 31 and SEQ.ID.NO. 33) and of 9 subtractive clones was assessed using a PCR and a RT-PCR assays (FIG. 2).

The PCR assays were carried out using as DNA template cDNAs obtained from a reverse transcription procedure on mRNAs extracted from salivary glands either of engorged or of unfed ticks.

Each PCR assay included pair of primers specific to each target subtractive or cDNAs full-length sequence. PCR assays were performed in a final volume of 50 μl containing 20 pM primers, 0.2 mM deoxynucleotide (dATP, dCTP, dGTP and dTTP; Boehringer Mannheim GmbH, Mannheim, Germany), PCR buffer (10 mM TrisHC1,50 mM KCI, 2.5 mM. MgC12, pH 8.3) and 2.5 U of Taq DNA polymerase (Boehringer Mannheim GmbH, Mannheim, Germany).

DNA samples were amplified for 35 cycles under the following conditions: 94° C. for 1 min., 72° C. for 1 min. and 64° C. for 1 min, followed by a final elongation step of 72° C. for 7 min.

The RT-PCR assay was carried out on the 5 selected full-length cDNA clones and on 5 cDNA subtractive clones. The mRNAs used as template in the reverse transcription assay was extracted from salivary glands of engorged and unfed I. ricinus ticks. The reverse transcription assays were performed using a specific primer (that target one the selected sequences) and the “Thermoscript Reverse transcriptase” (Life technologies, Rockville, Md., USA) at 60° C. for 50 min. Each PCR assay utilised the reverse transcription specific primer and an another specific primer. The PCR assays were performed in a final volume of 50 μl containing 1 μM primers, 0.2 mM deoxynucleotide (dATP, dCTP, dGTP and dTTP; Boehringer Mannheim GmbH, Mannheim, Germany), PCR buffer (10 mM Tris HCI, 50 mM KCI, 2.5 mM MgCl₂, pH 8.3) and 2.5 U of Expand High Fidelity polymerase (Roche, Bruxelles, Belgium). Single stranded DNA samples were amplified for 30 cycles under the following conditions: 95° C. for 1 min., 72° C. for 30 sec. and 60° C. for 1 min, followed by a final elongation step of 72° C. for 7 min.

The FIG. 2 shows that the expression of the selected sequences is induced in salivary glands of 5 day engorged ticks, except for the sequence 31 that is expressed at a similar level in salivary glands of engorged and unfed ticks. The expression of the other mRNAs could be either induced specifically or increased during the blood meal.

### Example 6

### Expression of Recombinant Proteins in Mammal Cells

The study of the properties of isolated sequences involves the expression thereof in expression systems allowing large amounts of proteins encoded by these sequences to be produced and purified.

The DNA sequences of the 5 selected clones (SEQ.ID.NO. 7, SEQ.ID.NO. 17, SEQ.ID.NO. 26, SEQ.ID.NO. 31 and SEQ.ID.NO. 33) were transferred into the pCDNA3.1 His/V5 expression vector. Said vector allows the expression of heterologous proteins fused to a tail of 6 histidines as well as to the V5 epitope in eucaryotic cells. The different DNAs were produced by RT-PCR by using primers specific to the corresponding clones. These primers were constructed so as to remove the stop codon of each open reading frame or phase in order to allow the protein to be fused to the 6×HIS/Epitope V5 tail. In addition, the primers contained restriction sites adapted to the cloning in the expression vector. Care was taken to use, when amplifying, a high fidelity DNA polymerase (Pfu polymerase, Promega).

The transient expression of the SEQ.ID.NO. 17 and SEQ.ID.NO. 26 recombinant proteins was measured after transfection of the SEQ.ID.NO. 17 and SEQ.ID.NO. 26-pCDNA3.1-His/V5 constructions in COS1 cells, using Fugen 6 (Boehringer). The protein extracts of the culture media corresponding to times 24, 48 and 72 hours after transfection were analysed on acrylamide gel by staining with Coomassie blue or by Western blot using on the one hand an anti-6× histidine antibody or on the other hand Nickel chelate beads coupled to alkaline phosphatase.

These analyses showed the expression of said proteins in the cell culture media.

### Example 7

### Expression of Proteins in E. coli

7.1. Insertion of Coding Sequences into the pMAL-C2E Expression Vector.

Proteins fused with the Maltose-Binding-Protein (MBP) were expressed in bacteria by using the pMAL-C2E (NEB) vector. The protein of interest then could be separated from the MBP thanks to a site separating the MBP from the protein, said site being specific to protease enterokinase.

In order to express optimally the 5 sequences examined, using the pMAL-C2E vector, PCR primer pairs complementary to 20 bases located upstream of the STOP codon and to 20 bases located downstream of the ATG of the open reading frame or phase were constructed. The amplified CDNA fragments only comprise the coding sequence of the target mRNA provided with its stop codon. The protein of interest was fused to MBP by its N-terminal end. On the other hand, since these primers contained specific restriction sites specific to the expression vector, it was possible to effect direct cloning of the cDNAs. The use of Pfu DNA polymerase (Promega) made it possible to amplify the cDNAs without having to fear for errors introduced into the amplified sequences.

The coding sequences of clones SEQ.ID.NO. 7, SEQ.ID.NO. 17, SEQ.ID.NO. 26 and SEQ.ID.NO. 31 were reconstructed in that way. Competent TG1 cells of E. coli were transformed using these constructions. Enzymatic digestions of these mini-preparations of plasmidic DNA made it possible to check that the majority of clones SEQ.ID.NO. 7, SEQ.ID.NO. 17, SEQ.ID.NO. 26 and 31 -p-MALC2-E effectively were recombinant.

7.2. Expression of Recombinant Proteins.

Starting from various constructions cloned in TG1 E. coli cells, the study of the expression of recombinant proteins fused with MBP was initiated for all sequences of interest (i.e. SEQ.ID.NO. 7, SEQ.ID.NO. 17, SEQ.ID.NO. 26 and SEQ.ID.NO. 33) except for SEQ.ID.NO. 31. The culture of representative clones of SEQ.ID.NO. 7, SEQ.ID.NO. 17, SEQ.ID.NO. 26 and SEQ.ID.NO. 33 as well as negative controls (non recombinant plasmids) were started to induce the expression of recombinant proteins therein. These cultures were centrifuged and the pellets were separated from the media for being suspended in 15 mM pH7.5 Tris and passed through the French press. The analysis of these samples on 10% acrylamide gel coloured with Coomassic blue or by Western Blot using rabbit anti-MBP antibodies, showed the expression of recombinant proteins SEQ.ID.NO. 7 (˜50 kDa), SEQ.ID.NO. 17 (˜92 kDA), SEQ.ID.NO. 26 (˜80 kDA) and SEQ.ID.NO. 31 (−67 kDa).

### Example 8

### Production of Antibodies

The SEQ.ID.NO. 7, SEQ.ID.NO. 17 and SEQ.ID.NO. 26 protein were injected into groups of 4 mice with the purpose of producing antibodies directed against said proteins. The antigens were firstly injected with the complete Freund adjuvant. Two weeks later, a recall injection was made with incomplete Freund adjuvant. The sera of mice injected with SEQ.ID.NO. 17 provided positive tests for anti-MBP antibodies.

### Example 9

### SEQ.ID.NO. 17: Protein Characterization

The SEQ.ID.NO. 17 protein sequence is homologous to various metallopeptidases inhibiting the platelet aggregation. Subsequently, immunological tests were performed with mammalian cells culture medium expressing the recombinant SEQ.ID.NO. 17/His sequence. These experiments were performed using culture medium of expressing SEQ.ID.NO. 17/His CHO-K1 cells (concentration about 75 nM). The same culture medium of cells non-expressing the recombinant protein was used as negative control (NEG). In short, we showed that SEQ.ID.NO. 17/His protein inhibits the production of some cytokines (IFN-γ, IL-10, IL-6, TNF-α) when human PBMC's are stimulated by PPD that activates antigen presenting cells in particular. Moreover, the SEQ.ID.NO. 17 protein seems to have immunological properties by inducing the proliferation of lymphatic T cells. Finally, genetic immunization experiments suggest that SEQ.ID.NO. 17 generates an immune response in mice capable of ejecting or destroying ticks.

Amino Acid and Nucleic Acid Sequence Analysis

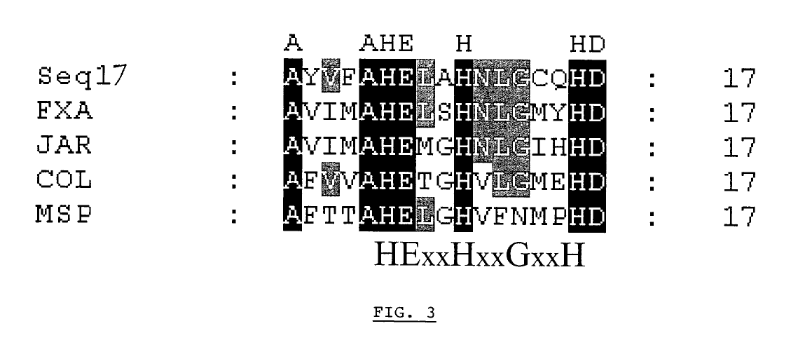

The SEQ.ID.NO. 17 cDNA and deduced amino acid sequence were analysed by the tFasta, Blastp, and Motifs algorithms of the GCG Wisconsin package software. The size of the deduced amino acid sequences from this coding sequence is 489 bp. SEQ.ID.NO. 17 is homologous to a mouse secretory metalloprotease containing thrombospondin motifs (E=3.10⁻⁷) (FIG. 3). As one approach to determining whether this protein is secreted, the deduced amino acid sequence of the cDNA was analysed for the presence of a signal peptide sequence. This cDNA encodes putative secretory signal peptide motifs (Table 3).

TABLE 3
Analysis of SEQ. ID. NO. 17 amino-acid sequence. Complete SEQ. ID. NO. 17 cDNA sequence
was compared to EMBL/GenBank databases using tFasta or Blastp algorithm. The
SEQ. ID. NO. 17 sequence was analysed for the presence of either motives (motifs algorithm)
or a specific signal peptide sequence (MeGeoch analysis).

| | | | | | Signal |
|---|---|---|---|---|---|
| | Full-length sequences similarity | tFasta/Blastp | ORF | | peptide |
| Clone | to databases | Scores^{a} | (aa) | Motives | scores^{b} |
| Seq17 | Mouse mRNA for secretory protein | 0,002/6.10⁻⁷ | 489 | Metallopeptidase | 7,9/S |
| | containing thrombospondin motives | | | | |
| | [D67076] | | | | |

^{a}No score (N)
^{b}Succeed (S) and Failed (F)
^{c}EMBL/GenBak accession number are indicated in brackets.

SEQ.ID.NO. 17 Detection in I. ricinus Salivary Glands

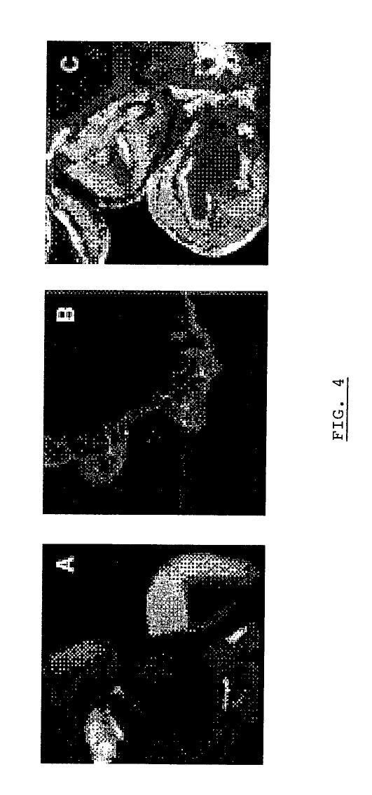

Confocal microscopy showed that SEQ.ID.NO. 17 was expressed in the salivary glands of female I. ricinus ticks being fed during 5 days. The protein was detected, thanks to anti SEQ.ID.NO. 17/His serum, under light and on the acini external surface (FIG. 4). On the contrary, the protein was not found in the salivary glands of unfed ticks.

Characterization of SEQ.ID.NO. 17 Immuno-Modulatin Properties.

ELISA tests were performed to study the modulation of cytokines expression by human PBMC's incubated with SEQ.ID.NO. 17/His culture medium and stimulated with various activators. In each test, proliferation was negatively controlled by stimulating the cells with the activator only. What's more, SEQ.ID.NO. 17/His and NEG culture media were shown not to be toxic to PBMC's, because they do not impair their viability. When stimulating PBMC's by PPD, SEQ.ID.NO. 17/His culture medium inhibited the expression of the various cytokines (IFN-γ, IL-10, IL-6, TNF-α) except IL-8 and IL-1 (Table 4). In addition, SEQ.ID.NO. 17/His culture medium had not impact on the cytokines production by the PBMC's stimulated by the other activators. NEG culture medium had no relevant effect on the cytokines production.

TABLE 4
Production of cytokines by PBMC's co-stimulated by SEQ. ID. NO. 17/
His culture medium and PPD. Expression of cytokines is inhibited (−) or
unchanged (/). Values represent percentages of expression calculated in
comparison to cells stimulated by NEG extract.

| Cytokines | PPD stimulation | % of expression* |
|---|---|---|
| IFN-γ | − | 18 |
| IL-6 | − | 37 |
| TNF-α | − | 25 |
| IL-10 | − | 32 |
| IL-1β | / | 97 |

Proliferation of Cells from Draining Lymph Nodes

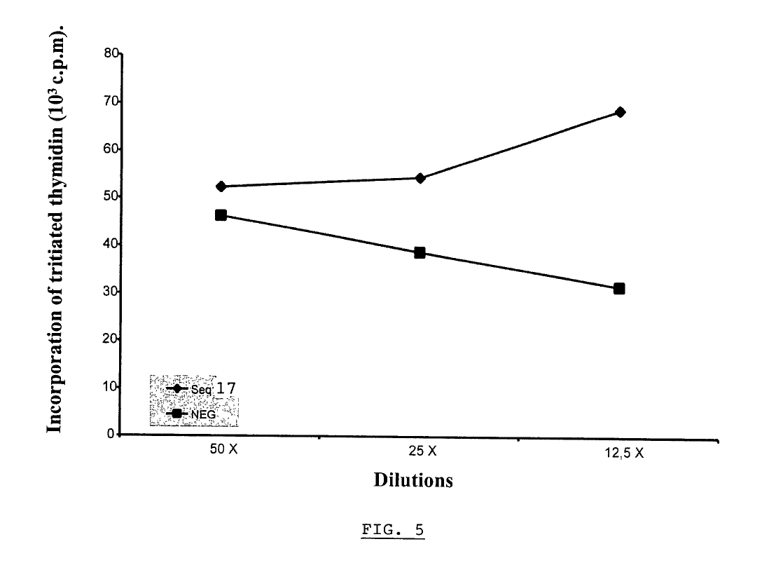

SEQ.ID.NO. 17 immunogenicity was studied in proliferation tests of cells from draining lymph nodes of Balb/C mouse pre-infested with I. ricinus nymphae. The lymph nodes draining the biting site were isolated 9 days after the infestation start. The lymphatic cells were stimulated by different dilutions of culture medium containing SEQ.ID.NO. 17/His or the negative control (NEG). The cell proliferation was assessed by measuring the incorporation of tritiated thymidin during 72 hours (FIG. 5). SEQ.ID.NO. 17/His culture medium induced cell proliferation with a dose effect. Indeed, cell proliferation increases when the cells are stimulated by growing concentrations of SEQ.ID.NO. 17/His culture medium. NEG culture medium, used with different dilutions, slightly inhibits the cell proliferation (FIG. 5). This result shows that SEQ.ID.NO. 17/His protein has immunogenic properties by specifically inducing the proliferation of lymphatic cells whereas negative control slightly inhibits the proliferation of these cells.

Genetic Immunization Experiment

Ticks's saliva contains proteins playing a major role in the blood meal completion. Moreover, different SAT (saliva-activated transmission) factors, of protic origin too, ease the transmission of pathogens. These SAT factors could be identical to the factors modulating the host defence mechanisms and allowing the tick to complete its blood meal.

An in vivo study of the protein and the assessment of its vaccine potentialities was made possible through a genetic immunization experiment on mice. The “vaccination by DNA” method consists in injecting in the mouse tibialis muscle plasmids that are vectors of an heterologous gene under control of a functional promoter in mammalian cells, such as hCMV promoter. When a protective immune response is being developed against an antigen expressed in that way, it frequently implicates mechanisms of cellular and humoral immunity. On the contrary, immunization by purified protein injection only induces humoral immunity mechanisms. These mechanisms induce specific antibody production; they do not always enable protective immune response to take place though. This genetic immunization experiment was performed in order to induce an immune response neutralizing SEQ.ID.NO. 17 protein activity when that protein is naturally delivered to the vaccinated host by the tick.

The genetic immunization experiment was performed on 2 groups of 3 C57/Black mice and 2 Balb/C mice each. These mice were immunized by 4 injections of pcDNA3.1-V5/His vector carrying SEQ.ID.NO. 17 coding DNA at three weeks interval. The first group was immunized against SEQ.ID.NO. 17/His protein whereas the second group, which was the negative control group, only received pCDNA3.1 -V5/His vector. IgG titres of collected sera were tested by ELISA on SEQ.ID.NO. 17/MBP purified recombinant protein (Table 4). The results show that mice 1-1B and 1-2B developed much higher specific anti-SEQ.ID.NO. 17 antibodies than C57/Black mice. Indeed, the 3 C57/Black mice from Group I (SEQ.ID.NO. 17) did not developed antigen specific antibodies.

Subsequently, these mice were infested each with 15 I. ricinus nymphae collected in the countryside of Neuchätel (Switzerland), which is an endemic zone for B. burgdorferi. Fed nymphae were identified and weighed after their blood meal. The development of resistance to tick infestation was analysed by comparing feeding time and average weight of the various tick groups at the end of their meal (Table 5). For Balb/C mouse with a high anti-SEQ.ID.NO. 17 antibody titre (mouse 1.2B) all ticks were dead on the second day of the blood meal (Table 4). On the contrary, ticks were normally fed on the second Balb/C mouse (1.1 B) as well as on that group C57/Black mice (1.1C, 1.2C and 1.3 C). The results obtained with mouse 1.2B suggest that SEQ.ID.NO. 17 protein generates an immune response in mice capable of ejecting or destroying ticks. The SEQ.ID.NO. 17 protein and its encoding nucleotide sequence or a pharmaceutical composition comprising them can be used as for the treatment and/or the prevention of cardiovascular diseases especially cardiovascular disesases caused by platelet aggregation. Examples of said cardiovascular diseases are thromboembolic disease or thrombotic pathologic condition in mammal, which are selected from the group consisting of ischemic disease, ischemic stroke, ischemic cerebral infarction, acute myocardial infarction, chronic ischemic heart disease, ischemic disease of an organ other than myocardium or a region of the brain, venous thromboembolism, arterial or venous thrombosis, pulmonary embolism, restenosis following coronary artery bypass surgery or following percutaneous transluminal angioplasty of coronary artery and other diseases of ischemic origin including grangraine, Raynaud disease or hypertension (systemic hypertension, essential hypertension, maligant hypertension, renal hypertension and pulmonary hypertension).

TABLE 5
Genetic immunization experiment. Analysis of average weight and feeding
time of ticks.

| | | | | | |
|---|---|---|---|---|---|
| | | Average | | | |
| | | weight | | Infection by | |
| Antigen | Mouse | (mg) | IgG titre | Borrelia | Observation |

| | | | | | |
|---|---|---|---|---|---|
| Seq16 | 1.1B | 3.56 | 117 | + | * |
| | 1.2B | 0 | 419 | − | |
| | 1.1C | 3.91 | 0 | ++ | |
| | 1.2C | 3.41 | 0 | ++ | |
| | 1.3C | 3.68 | 0 | (+)immobile | |
| Negative | 4.1B | 4.1 | − | ++ | dead mouse |
| | 4.2B | 2.68 | − | | |
| | 4.1C | 3.98 | − | − | |
| | 4.2C | 3.76 | − | − | |
| | 4.3C | 4.35 | − | − | |

In the different groups, mice XXB are Balb/C mice and mice XXC are C57/Black mice. The average weight is the average of the weight of each tick at the end of its blood meal. The IgG titre of each mouse was assessed by ELISA against recombinant proteins produced in bacteria. * Ear biopsy was revealed positive later than for C57/Black mice.

What is claimed is:

1. A polynucleotide obtained from tick salivary gland and presenting more than 75% identity with the nucleotide sequence SEQ.ID.NO. 17 or a sequence complementary thereto, or an active fragment thereof.

2. The polynucleotide of claim 1, which presents at least 80% identity with SEQ.ID.NO. 17 nucleotide sequence.

3. The polynucleotide of claim 1, which is at least 90% identical with SEQ.ID.NO. 17 nucleotide sequence.

4. The polynucleotide of claim 1, which is at least 95% identical with SEQ.ID.NO. 17 nucleotide sequence.

5. The polynucleotide of claim 1, which is at least 98-99% identical with SEQ.ID.NO. 17 nucleotide sequence.

6. The polynucleotide of claim 1, which is at least 99% identical with SEQ.ID.NO. 17 nucleotide sequence.

7. A polypeptide encoded by the polynucleotide of claim 1, or a biologically active fragment or portion thereof.

8. A polypeptide according to claim 7, modified by or linked to at least one substitution group, preferably selected from the group consisting of amide, acetyl, phosphoryl, and/or glycosyl groups.

9. The polypeptide of claim 7 in the form of a “mature” protein.

10. The polypeptide of claim 7 as part of a larger protein.

11. The polypeptide of claim 7 as part of a fusion protein.

12. The polypeptide of claim 7 further including at least one additional amino acid sequence which contains secretory or leader sequences, pro-sequences, sequences which help in purification such as multiple histidine residues, or additional sequences for stability during recombination protection.

13. A variant comprising a polynucleotide according to claim 1, a polypeptide encoded by the polynucleotide of claim 1, a biologically active fragment of a polypeptide encoded by the nucleotide of claim 1 or portion thereof.

14. The variant according to claim 13, which said polypeptide varies from the referent by conservative amino acid substitutions.

15. The variant according to claim 13 in which said polypeptide comprises at least one residue which is substituted with another residue of like characteristics.

16. The variant according to claim 15, in which said polypeptide comprises substitutions, wherein the substitutions are among Ala, Val, Leu and Ile; among Ser and Thr, among the acidic residues Asp and Glu; among Asn and Gln; among the basic residues Lys and Arg; or among aromatic residues Phe and Tyr.

17. The variant according to claim 13, in which the polypeptide comprises several amino acids which are substituted, deleted or added in any combination.

18. The variant according to claim 13, wherein said polypeptide comprises 5-10 amino acids which are substituted, deleted or added in any combination.

19. The variant according to claim 13, wherein said polypeptide comprises 1-5 amino acids which are substituted, deleted or added in any combination.

20. The variant according to claim 13, wherein said polypeptide comprises 1-2 amino acids which are substituted, deleted or added in any combination.

21. The variant according to claim 13, wherein said polypeptide is a naturally occurring allelic variant of a Ixodes ricinus salivary gland polypeptide present in Ixodes ricinus salivary glands.

22. A vector comprising at least one element selected from the group consisting of:
a) a polynucleotide obtained from tick salivary gland and presenting more than 75% identity with the nucleotide sequence SEQ.ID.NO. 17 or a sequence complementary thereto, or an active fragment thereof,
b) a polypeptide encoded by the polynucleotide of a) above, a biologically active fragment of a polypeptide encoded by the nucleotide of a) above or portion thereof; and
c) a variant comprising a polynucleotide according to a) above, a polypeptide encoded by the polynucleotide of a) above, a biologically active fragment of a polypeptide encoded by the nucleotide of a) above or portion thereof.

23. A cell transfected or comprising the vector according to claim 22.

24. An inhibitor directed against an element selected from the group consisting of:
a) a polynucleotide obtained from tick salivary gland and presenting more than 75% identity with the nucleotide sequence SEQ.ID.NO. 17 or a sequence complementary thereto, or an active fragment thereof,
b) a polypeptide encoded by the polynucleotide of a) above, a biologically active fragment of a polypeptide encoded by the nucleotide of a) above or portion thereof; and
c) a variant comprising a polynucleotide according to a) above, a polypeptide encoded by the polynucleotide of a) above, a biologically active fragment of a polypeptide encoded by the nucleotide of a) above or portion thereof.

25. The inhibitor according to claim 24, which is an antibody or an hypervariable portion thereof.

26. A hybridoma cell line expressing the inhibitor according to claim 25.

27. A pharmaceutical composition comprising an adequate pharmaceutical carrier and an element selected from the group consisting of:
a) a polynucleotide obtained from tick salivary gland and presenting more than 75% identity with the nucleotide sequence SEQ.ID.NO. 17 or a sequence complementary thereto, or an active fragment thereof,
b) a polypeptide encoded by the polynucleotide of a) above, a biologically active fragment of a polypeptide encoded by the nucleotide of a) above or portion thereof;
c) a variant comprising a polynucleotide according to a) above, a polypeptide encoded by the polynucleotide of a) above, a biologically active fragment of a polypeptide encoded by the nucleotide of a) above or portion thereof;
d) a vector comprising at least one element selected from the group consisting of a polynucleotide according to a) above, a polypeptide encoded by the polynucleotide of a) above, a biologically active fragment of a polypeptide encoded by the nucleotide of a) above or a portion thereof, and a variant comprising a polynucleotide according to a) above, a polypeptide encoded by the polynucleotide of a) above, a biologically active fragment of a polypeptide encoded by the nucleotide of a) above or portion thereof;
e) a cell comprising the vector of d) above;
f) an inhibitor directed against an element selected from the group consisting of a polynucleotide according to a) above, a polypeptide encoded by the polynucleotide of a) above, a biologically active fragment of a polypeptide encoded by the nucleotide of a) above or a portion thereof, and a variant comprising a polynucleotide according to a) above, a polypeptide encoded by the polynucleotide of a) above, a biologically active fragment of a polypeptide encoded by the nucleotide of a) above or portion thereof; and
g) a mixture of any of a)-f) above.

28. The pharmaceutical composition according to claim 27 for inducing lymphatic cells proliferation.

29. The pharmaceutical composition according to claim 27 for the treatment of the prevention of cardiovascular disease.

30. An immunological composition or vaccine for inducing an immunological response in a mammalian host to a tick salivary gland polypeptide which comprises at least one element of the group consisting of:
a) a tick salivary gland polynucleotide presenting more than 75% identity with the nucleotide sequence SEQ.ID.NO. 17 or a sequence complementary thereto, or an active fragment thereof,
b) a tick salivary gland polypeptide encoded by the polynucleotide of a) above, a biologically active fragment of a polypeptide encoded by the nucleotide of a) above or portion thereof;
c) a variant comprising a polynucleotide according to a) above, a polypeptide encoded by the polynucleotide of a) above, a biologically active fragment of a polypeptide encoded by the nucleotide of a) above or portion thereof,
d) epitope-bearing fragments, analogs, outer-membrane vesicles or cells (attenuated or otherwise) of components a) or b) or c); and
e) possibly a carrier.

31. A method for treating or preventing a disease affecting a mammal, said method comprising the step of administrating to said mammal a sufficient amount of the pharmaceutical composition according claim 27 or the immunological composition or vaccine according to claim 30, in order to prevent or cure either the transmission of pathogenous agents by tick, especially by Ixodes ricinus, or the symptoms of diseases induced by tick or pathogenous agents transmitted by tick.

32. A method for treating or preventing a disease affecting a mammal, said method comprising the step of administrating to said mammal a sufficient amount of the pharmaceutical composition according to claim 27 or the immunological composition or vaccine according to claim 30, in order to induce lymphatic cells proliferation.

33. A method for treating or preventing a cardiovascular disease affecting a mammal, said method comprising the step of administering to said mammal a sufficient amount of the pharmaceutical composition according to claim 27.

34. A diagnostic kit for detecting a disease or susceptibility to a disease induced or transmitted by tick, especially Ixodes ricinus, which comprises:
a) the tick salivary gland polynucleotide, according to claim 1, or an active fragment thereof;
b) a nucleotide sequence complementary to that of a);
c) a tick salivary gland polypeptide encoded by the polynucleotide of a) above, a biologically active fragment of a polypeptide encoded by the nucleotide of a) above or portion thereof;
d) a variant comprising a polynucleotide according to a) above, a polypeptide encoded by the polynucleotide of a) above, a biologically active fragment of a polypeptide encoded by the nucleotide of a) above or portion thereof,
e) an inhibitor directed against the polynucleotide of a) above, the polypeptide of c) above, or the variant of d) above; and
f) a phage displaying an antibody according to e) above, whereby a), b), c), d), or e) may comprise a substantial component.
